(19) United States
(12) Patent Application Publication
(10) Pub. No.: US 2025/0001154 A1
(43) Pub. Date: Jan. 2, 2025

(54) SINGLE INTRAVENOUS T-SITE AND METHOD OF ADMINISTRATION

(71) Applicant: Nexus Medical, LLC, Lenexa, KS (US)

(72) Inventors: W. Cary Dikeman, Leawood, KS (US); Josh Hughey, Leawood, KS (US); Taylor Wilson, Lee’s Summit, MO (US)

(21) Appl. No.: 18/215,542

(22) Filed: Jun. 28, 2023

(51) Int. Cl. A61M 39/02

(52) U.S. Cl. CPC A61M 39/0247; A61M 2039/0258; A61M 2039/0264; A61M 2039/0288; A61M 2039/064

(57) ABSTRACT

A T-site connector has a T-site body, which includes a primary luer body and a tube bond stem, a septum, and an externally threaded cap. The T-site body includes a septum housing at a proximal end of the T-site body. The T-site body also includes the tube bond stem extending outwardly from the primary luer body. The internally threaded collar is secured such that the internally threaded collar is free-spinning to rotatably secure to a catheter. The septum has a slit for receipt of a cannula therethrough. The externally threaded cap is configured to secure to the septum housing of the T-site body with the septum therein. The externally threaded cap is configured to receive a proximal connection device thereon.

### BACKGROUND

### 1. FIELD

Embodiments of the invention relate generally to a medical connector for the removal of fluids from a patient and administration of medicaments to a patient. More specifically, embodiments of the invention are directed to a T-site connector to allow medical staff to efficiently access a patient's blood vessel.

### 2. RELATED ART

The insertion of peripheral intravenous catheters and peripheral blood collection needles are clinical practices commonly performed by clinicians and phlebotomists. When patients have small, damaged, collapsed veins or other symptoms and conditions such as dehydration and low blood pressure, this can further complicate the insertion of these devices into the patient's blood vessels.

The average hospitalized patient will encounter a stainless-steel needle placement or insertion (“stick”) every time a physician orders a blood test. This can occur as frequently as once an hour for arterial blood gas samples or more routinely twice a day. The standard procedure for blood sampling involves using a stainless-steel needle to enter or stick a selected vein in the patient's hand, forearm, or antecubital fossa.

Blood sampling is labor intensive and time consuming for clinician and phlebotomist. A single needle stick blood sampling procedure can take between 3 to 20 minutes for each blood sample. Blood collection is also painful and a major source of patient anxiety and discomfort. The patient's pain, discomfort and anxiety are further elevated when difficulty in finding a proper vein for blood sampling results in multiple needle sticks. Furthermore, multiple sticks also involve additional material expense as well as additional clinician and phlebotomist time.

A particular group of patients may additionally be defined to have Difficult Intravenous Access (DIVA), which is very few or no superficial vein access. In some cases, ultrasound may be used to assess the patient's vein in order to find a deeper more viable source for blood collection.

In most hospitalized patients (>90%), peripheral IV catheters are commonly used for the infusion of IV fluids, medications, drugs, and blood. Peripheral IV catheters, when placed into a larger vein in the forearm, may have a faster and higher volumetric blood flow than the peripheral IV catheter placed in the hand or wrist. When hospitalized, patients may use an anti-reflux needleless connector to protect the catheter lumen from unintentional blood aspiration, which can also lead to blood clots.

Due to the pain, trauma, anxiety and discomfort for the patient and the time-consuming nature of finding a proper functioning vein for an efficient blood draw, there is a need for a well-placed peripheral IV catheter that can be used successfully for blood collection.

### SUMMARY

Embodiments of the invention solve the above-mentioned problems by providing a single intravenous T-site connector, system, method of administration, blood sampling, and collection. The single intravenous T-site connector is configured to be used with a standard peripheral IV catheter that is inserted into the patient's forearm. The T-site connector is secured to the catheter to allow for at least one needleless connector to be secured to the primary access port for the injection and infusion of medicaments and the withdrawal of blood via the T-Site connector while connected to the patient's Peripheral IV catheter, which is inserted in the patient's forearm or other viable vein. This allows the catheter to be used for multiple purposes.

In some aspects, the techniques described herein relate to a T-site connector configured to fluidly attach a syringe with a blood vessel of a patient, the T-site connector including: a T-site body including: a septum housing at a proximal end having a primary luer body extending distally therefrom and defining a primary fluid passageway; and a tube bond stem extending axially from the primary luer body and defining a secondary fluid passageway therein, the primary fluid passageway and the secondary fluid passageway being fluidly coupled at a central fluid chamber; an externally threaded cap including: a proximal face disposed at a proximal end of the externally threaded cap, the proximal face including a bore therethrough, the bore defining a first diameter; an external wall extending distally from the proximal face and having one or more external threads disposed circumferentially therearound; and an inner flange extending distally from the proximal face and within the external wall, the inner flange defining a second diameter being larger than the first diameter; and a septum housed within the inner flange and the septum housing, the septum including a slit therethrough, the slit being accessible through the bore.

In some aspects, the techniques described herein relate to an injection apparatus, including: a T-site connector and a locking cannula, the T-site connector including: a T-site body, a septum, and an externally threaded cap, the T-site body including a septum housing fluidly coupled to a primary luer body and a tube bond stem; the septum including a swabbable face having a slit therethrough and extending to a distal wall, the septum having a pre-use position and an in-use position; the externally threaded cap secured to the septum housing of the T-site body and including a proximal face and an external wall extending distally therefrom, the external wall including one or more external threads disposed thereon, wherein the septum is retained between portions of the externally threaded cap and the septum housing; the locking cannula including: a cannula having a proximal annular base connected to a cannula tip and defining a cannula fluid path therein; and an internally threaded collar segment receiving the cannula tip therein and being rotatable therearound, wherein the internally threaded collar segment includes one or more internal threads configured to rotatably attach to the one or more external threads of the externally threaded cap.

In some aspects, the techniques described herein relate to a system for controlling flow of liquids to and from a patient, including: a T-site connector including: a T-site body extending longitudinally between a proximal end and a distal end, wherein the proximal end includes a septum housing and the distal end includes a primary luer body having a tube bond stem disposed therebetween and extending axially in relation to the longitudinal direction; a septum disposed within the septum housing, the septum including a slit therethrough; an externally threaded cap disposed at the proximal end, the externally threaded cap including: a proximal face having a bore therethrough; an external wall extending distally from the proximal face and having one or more external threads disposed therearound, wherein the externally threaded cap is secured to the septum housing, thereby retaining the septum within the septum housing; and a first internally threaded collar disposed at the primary luer body and being rotatable therearound; a locking cannula including: a cannula; and a second internally threaded collar receiving a portion of the cannula therethrough, the second internally threaded collar including one or more internal threads configured to receive the one or more external threads of the externally threaded cap.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Embodiments of the present invention are described in detail below with reference to the attached drawing figures, wherein:

FIG. 1 depicts a proximal perspective view of a T-site connector of some embodiments;

FIG. 2 depicts a side view of the T-site connector of FIG. 1, in some embodiments;

FIG. 3 depicts an end view of the T-site connector of FIG. 1 as shown from a proximal side, in some embodiments;

FIG. 4 illustrates a vertical cross-sectional view through the line 4-4 of FIG. 3, in some embodiments;

FIG. 5 depicts a proximal perspective view of a T-site body of the T-site connector, in some embodiments;

FIG. 6 depicts a side view of the T-site body of FIG. 5, in some embodiments;

FIG. 7 depicts some embodiments of an end view of the T-site body of FIG. 5 as shown from a proximal side;

FIG. 8 illustrates a vertical cross-sectional view through the line 8-8 of FIG. 7, in some embodiments;

FIG. 9 depicts a proximal perspective view of an externally threaded cap of the T-site connector, in some embodiments;

FIG. 10 depicts a side view of the externally threaded cap of FIG. 9, in some embodiments;

FIG. 11 depicts some embodiments of an end view of the externally threaded cap of FIG. 9 as shown from a proximal side;

FIG. 12 illustrates a vertical cross-sectional view through the line 12-12 of FIG. 11, in some embodiments;

FIG. 13 depicts a perspective view of a symmetrical slit septum of the T-site connector, in some embodiments;

FIG. 14 depicts an end view of the slit septum of FIG. 13, in some embodiments;

FIG. 15 illustrates a vertical cross-sectional view through the line 15-15 of FIG. 14, in some embodiments;

FIG. 16 depicts some embodiments of a top view of an injection site system including the T-site connector and a lever-locking cannula prior to connection;

FIG. 17 depicts some embodiments of a top view of the injection site system of FIG. 16 with the T-site connector and the lever-locking cannula connected;

FIG. 18 depicts some embodiments a perspective view of a tube set that can be utilized with the T-site connector;

FIG. 19 illustrates some embodiments of a vertical cross-section view of a threaded-locking cannula utilized with the T-site connector and a syringe;

FIG. 20 depicts a proximal perspective view of a locking cannula shown from a proximal end, in some embodiments;

FIG. 21 depicts a distal perspective view of the locking cannula of FIG. 20 shown from a distal end, in some embodiments;

FIG. 22 depicts a proximal perspective view of a T-site connector of some embodiments;

FIG. 23 depicts a side view of the T-site connector of FIG. 22, in some embodiments;

FIG. 24 depicts an end view of the T-side connector of FIG. 22, as shown from a proximal side, in some embodiments;

FIG. 25 illustrates a vertical cross-sectional view through the line 25-25 of FIG. 24, in some embodiments;

FIG. 26 depicts a proximal perspective view of a T-side body of some embodiments;

FIG. 27 depicts a side view of the T-side body of FIG. 26, in some embodiments;

FIG. 28 illustrates a horizontal cross-sectional view through 28-28 of FIG. 27, in some embodiments;

FIG. 29 depicts a proximal perspective view of an externally threaded cap, in some embodiments;

FIG. 30 depicts a distal perspective view of the externally threaded cap of FIG. 29, in some embodiments;

FIG. 31 illustrates a vertical cross-sectional view of the externally threaded cap of FIGS. 29-30, in some embodiments;

FIG. 32 depicts a proximal perspective view of a septum, in some embodiments;

FIG. 33 illustrates a vertical cross-sectional view of the septum of FIG. 32;

FIG. 34 depicts a proximal view of a locking cannula, in some embodiments;

FIG. 35 illustrates a vertical cross-sectional view of the locking cannula of FIG. 34, in some embodiments;

FIG. 36 depicts a side view of the T-site connector of FIGS. 22-25 engaging the locking cannula of FIGS. 34-35, in some embodiments;

FIG. 37 illustrates some embodiments of a vertical cross-sectional view of the T-site connector engaging the locking cannula, as depicted in FIG. 36;

FIG. 38 depicts a side view of collar-locking cannula, in some embodiments;

FIG. 39 depicts a side view of the collar depicted in FIG. 38, in some embodiments;

FIG. 40 depicts some embodiments of the T-site connector connected to the collar-locking cannula depicted in FIGS. 38-39;

FIG. 41 depicts a perspective view of a catheter that may be used with some embodiments of the invention;

FIG. 42 is a flow diagram depicting exemplary steps of a method of installing a T-site connector;

FIG. 43 is a flow diagram depicting exemplary steps of a method of utilizing a T-site connector; and

FIG. 44 illustrates some embodiments of an intermediary tube segment configured to interact with portions of a T-site connector.

The drawing figures do not limit the invention to the specific embodiments disclosed and described herein. The drawings are not necessarily to scale, emphasis instead being placed upon clearly illustrating the principles of the invention.

### DETAILED DESCRIPTION

The subject matter of the invention is described in detail below to meet statutory requirements; however, the description itself is not intended to limit the scope of claims. Rather, the claimed subject matter might be embodied in other ways to include different steps or combinations of steps similar to the ones described in this document, in conjunction with other present or future technologies. Minor variations from the description below will be understood by one skilled in the art and are intended to be captured within the scope of the claimed invention. Terms should not be interpreted as implying any particular ordering of various steps described unless the order of individual steps is explicitly described.

The following detailed description of embodiments of the invention references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the present invention. The following detailed description is, therefore, not to be taken in a limiting sense. The scope of the invention is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, the present technology can include a variety of combinations and/or integrations of the embodiments described herein.

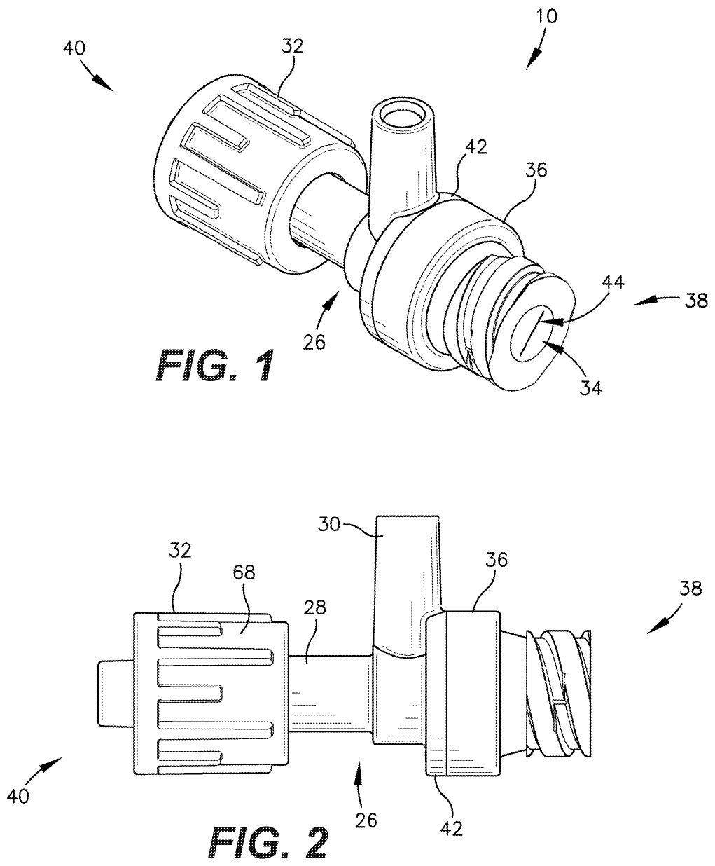

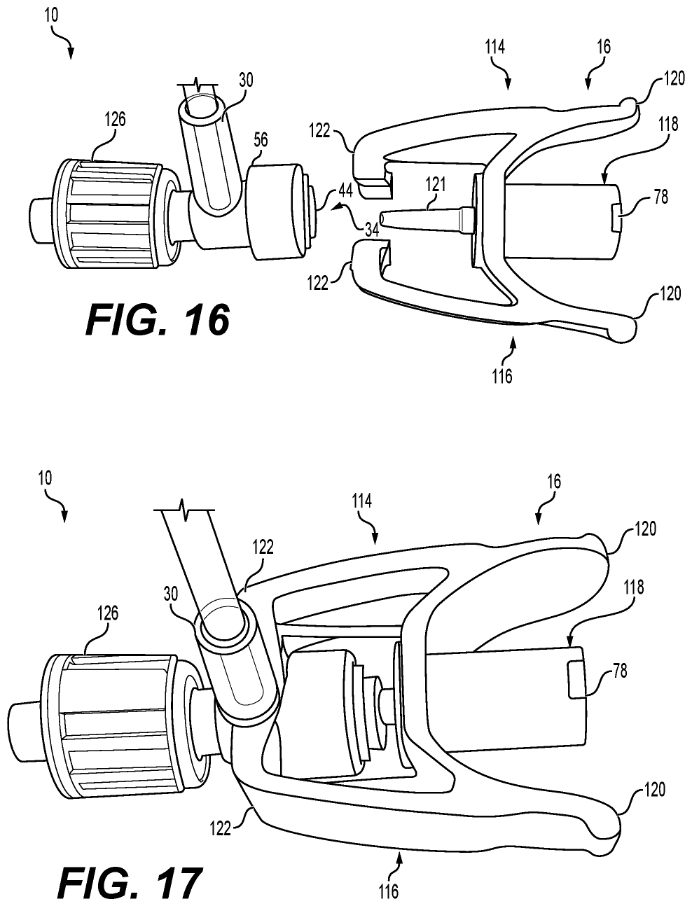

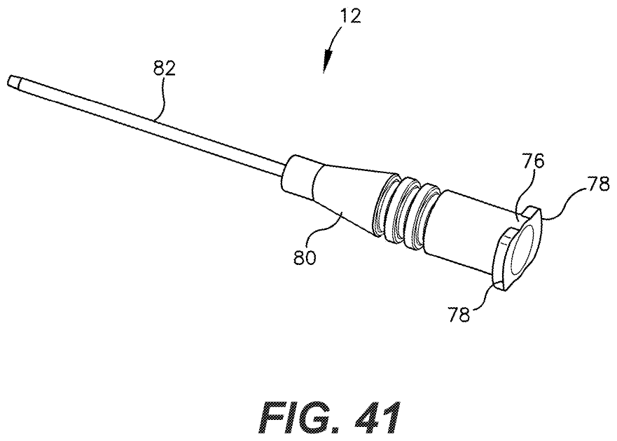

FIG. 1 illustrates a T-site connector 10 configured to be used with a catheter 12 (illustrated in FIG. 41), which may be inserted into the vein of a forearm of a patient. T-site connector 10 may be secured to catheter 12 to allow access for both administering and withdrawing fluids from a patient. For example, catheter 12 and T-site connector 10 may be used to inject medicaments into and/or draw blood out of the forearm of the patient. T-site connector 10 may be configured to allow for lever-locking cannula 16 (as illustrated in FIGS. 16) to be secured thereto. This provides increased versatility of T-site connector 10. For example, in some embodiments, T-site connector 10 may be secured to lever-locking cannula 16 and/or threaded-locking cannula 18. Threaded-locking cannula 18 may be configured to allow for a proximal medical device 128 to be used to attach to threaded-locking cannula 18, which can allow blood to be drawn from proximal medical device 128 and into a vacuum blood tube. As another example, a tube set 20 can be connected to threaded-locking cannula 18 and secured directly to T-site connector 10. Tube set 20 provides a plurality of access ports 22. Each access port 22 is configured to receive a blunt cannula (e.g., cannula 24), such that each access port 22 can be used to perform a specific function without impeding the function of the other access ports 22. As an example, access ports 22 may be used for drawing waste blood, blood sampling, and/or collection as well as flushing T-site connector 10.

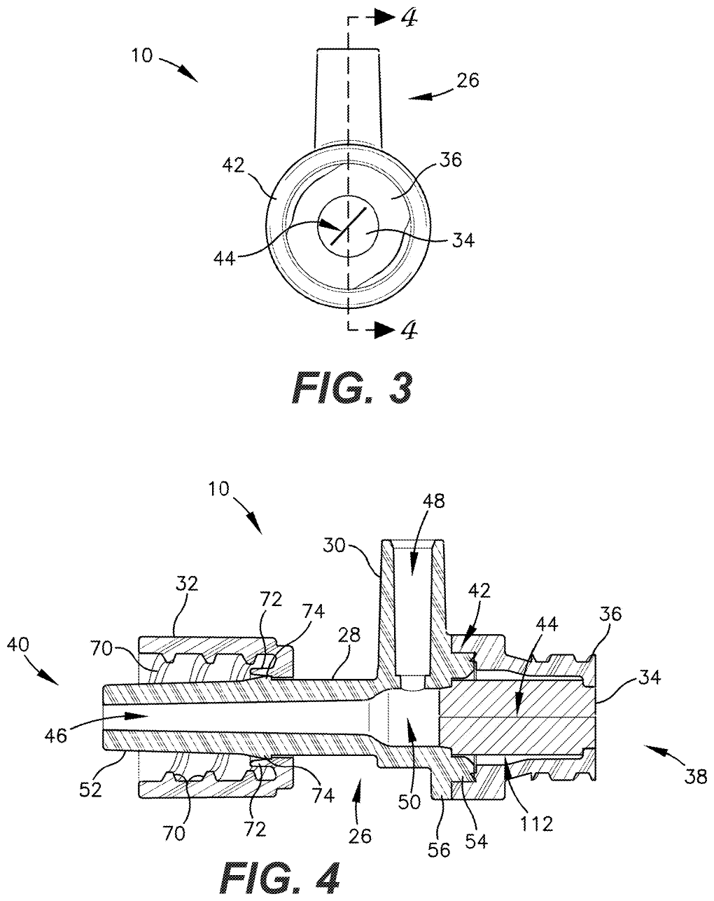

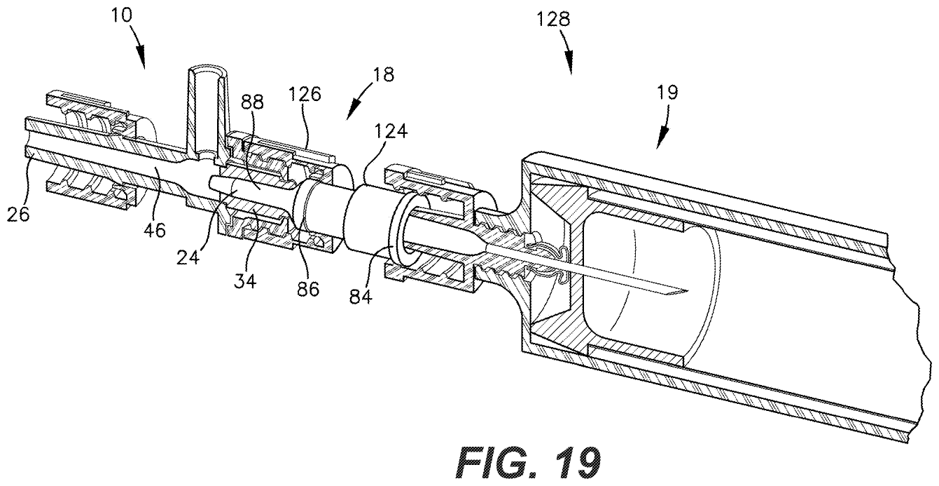

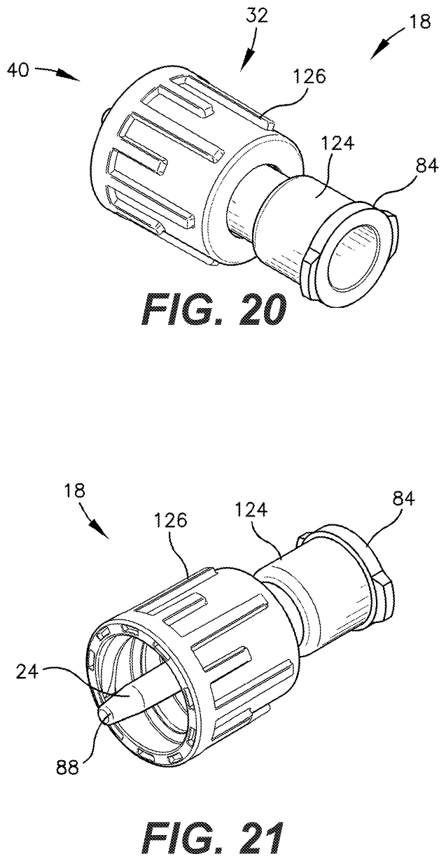

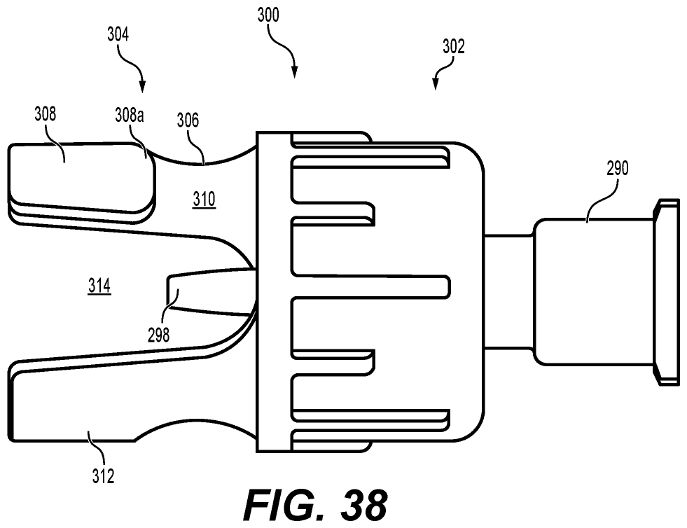

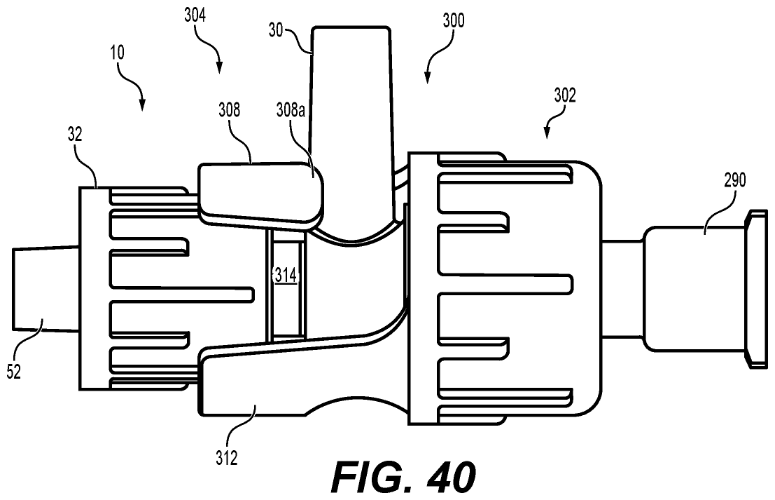

Turning to FIGS. 1-3, in some embodiments, T-site connector 10 includes a T-site body 26 comprising primary luer body 28, tube bond stem 30, internally threaded collar 32, slit septum 34, and externally threaded cap 36. T-site body 26 has a proximal end 38 and a distal end 40. In some embodiments, T-site body 26 includes internally threaded collar 32 located on or around primary luer body 28. The internally threaded collar 32 may be secured to primary luer body 28 at the distal end 40 of T-site body 26. Internally threaded collar 32 is secured such that internally threaded collar 32 is free-spinning relative to the primary luer body 28. Thus, internally threaded collar 32 is configured to be rotatably secured to catheter 12. In some embodiments, slit septum 34 has an axially formed slit 44 for receipt of a blunt cannula (e.g., cannula 24), lever-locking cannula 16 (see FIGS. 16-17), threaded-locking cannula 18 (see FIGS. 19-21), or collar-locking cannula (see FIGS. 38-40) therethrough. Slit septum 34 presents a generally cylindrical shape, in some embodiments. In embodiments, externally threaded cap 36 is configured to secure to septum housing 42 of T-site body 26 with the slit septum 34 therein. Externally threaded cap 36 may configured to receive cannula 24, lever-locking cannula 16, or threaded-locking cannula 18 thereon. For example, internally threaded cap segment, depicted in FIG. 20, may be rotatably secured to externally threaded cap 36. As such, cannula 24 may be inserted into and through slit septum 34, thereby allowing for the administration of fluids and/or blood collection from the patient.

Reference will be made herein to various orientations and dimensions of the T-site connector 10 and its components. As used herein, the T-site connector 10 and its components have longitudinal lengths that extend in a direction of a primary fluid passageway 46 (see FIGS. 4 and 8), as described below. The T-site connector 10 and components also present a transverse width that may be perpendicular to an axis of the primary fluid passageway 46. A distal end 40 of T-site connector 10 is configured to be oriented towards a patient when T-site connector 10 is in use by a medical professional (i.e., the end towards catheter 12), and proximal end 38 of T-site body 26 and components is configured to be oriented towards a medical professional administering fluids or collecting blood samples from the patient (i.e., the end towards the slit septum 34). Many of the T-site connector 10 components are generally cylindrical to present a radius from the axis of the primary fluid passageway 46 and to an outermost radial surface of the component when the T-site connector 10 is viewed from either its proximal end 38 or distal end 40. Therefore, reference to a “radial-most” surface refers to the surface that is bounded by the radius extending between the axis of primary fluid passageway 46 and the particular surface.

The various components of T-site connector 10 will now be discussed in more detail. It should be appreciated that T-site connectors of various embodiments may include any or all of the below-discussed components and may include additional components or the equivalents thereof. T-site connector 10 may also be used in connection with one of more of the below-discussed methods.

With respect to FIG. 4, T-site body 26 comprises primary luer body 28 having the primary fluid passageway 46 formed therethrough, such that primary fluid passageway 46 is the main fluid passageway through T-site connector 10. Primary luer body 28 includes septum housing 42 at the proximal end 38 of T-site body 26. In some embodiments, septum housing 42 receives a portion of slit septum 34 and securely connects to externally threaded cap 36, as described below.

In some embodiments, T-site body 26 comprises a molded rigid thermoplastic resin (such as MAKROLON® by BAYER®). In some embodiments, primary luer body 28 and tube bond stem 30 of T-site body 26 may be integrally formed to present a unitary, monolithic structure.

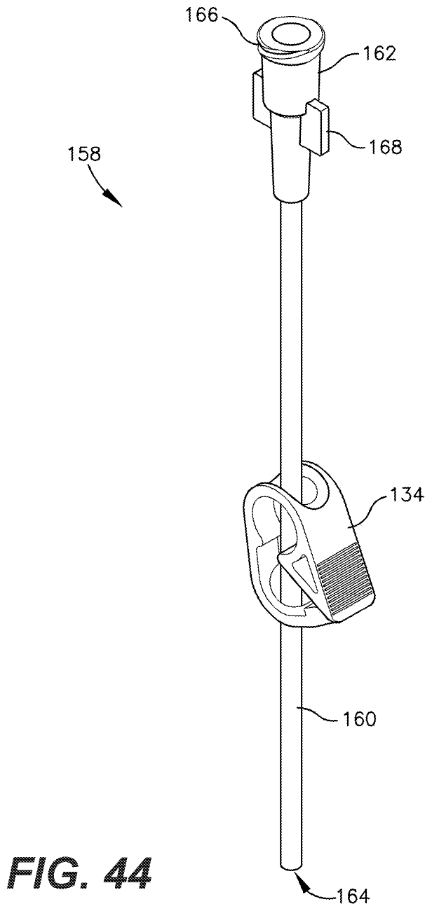

Tube bond stem 30 extends generally perpendicularly from primary luer body 28 and presents a secondary fluid passageway 48 (see FIGS. 4 and 8). Tube bond stem 30 provides a secondary access point for the administration of fluids to the patient via secondary fluid passageway 48. For example, in some embodiments tube bond stem 30 is configured to be secured to an intermediary tube segment 158 (illustrated in FIG. 44) for administration of fluids to the patient.

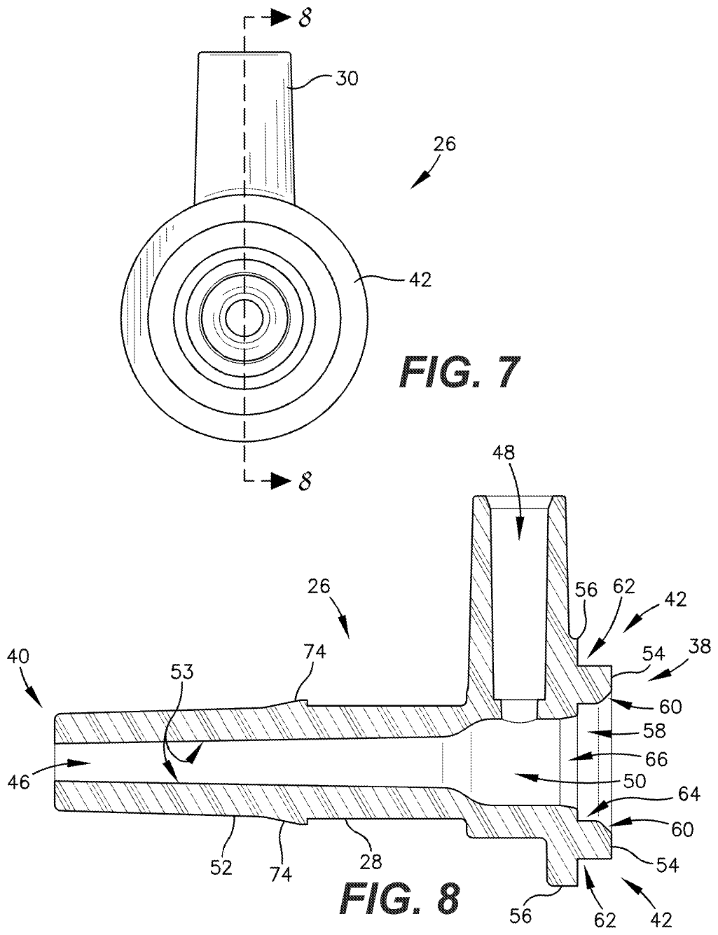

As shown in FIG. 8, which is a cross-sectional view from like 8-8 shown in FIG. 7, secondary fluid passageway 48 intersects primary fluid passageway 46 at a central fluid chamber 50, such that fluids administered via tube bond stem 30 flow to primary fluid passageway 46. Said another way, fluids administered through tube bond stem 30 travel through secondary fluid passageway 48, to central fluid chamber 50, and through primary fluid passageway 46. In use, the primary fluid passageway 46 may be fluidly connected to catheter 12 for administration of fluids intravenously, as discussed in more detail below.

Primary fluid passageway 46 extends the longitudinal length, or almost the entire longitudinal length, of primary luer body 28, as best illustrated in FIG. 8. T-site body 26 thus presents a longitudinal length extending between proximal end 38 and distal end 40. As seen in FIGS. 1-4, internally threaded collar 32 may be coupled to the distal end 40 of T-site body 26, and slit septum 34 is either integrally formed with or coupled to the proximal end 38 of T-site body 26, as described in more detail below. As shown in FIG. 8, primary fluid passageway 46 is an elongated opening formed through primary luer body 28 of the T-site body 26. In some embodiments, a male slip luer 52 of the primary fluid passageway 46 is generally conical in shape, such that a width of primary fluid passageway 46 tapers, i.e., narrows, from a proximal-most end of the primary fluid passageway 46 and towards a distal direction. That is, male slip luer 52 presents fluid passageway walls 53 that are inwardly angled in the distal direction. In some embodiments, the angle of the male slip luer 52 may be approximately 6-8 degrees.

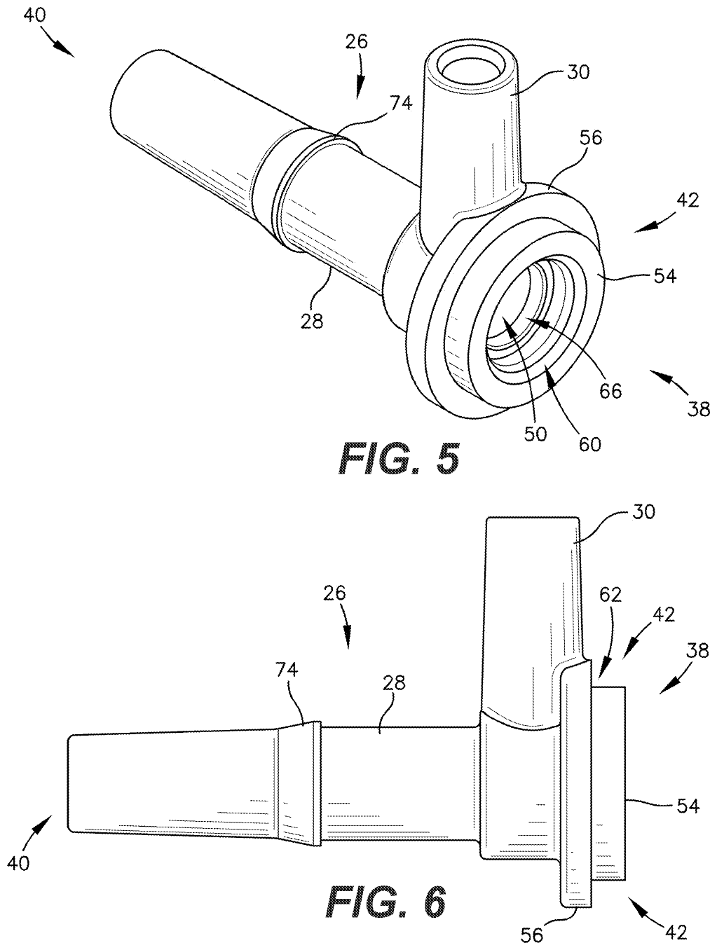

In some embodiments and as illustrated in FIGS. 5-6 and 8, septum housing 42 of T-site body 26 is configured to receive and retain slit septum 34 and the externally threaded cap 36 thereon. In some embodiments, septum housing 42 is a unitary component of the primary luer body 28. In some embodiments, septum housing 42 comprises an annular protrusion 54 extending proximally from primary luer body 28, a flange 56 extending radially from primary luer body 28, and a recess 58 extending into primary luer body 28. In some embodiments, annular protrusion 54 is configured to be secured to externally threaded cap 36. For example, in some embodiments annular protrusion 54 may be secured to externally threaded cap 36 via ultrasonic welding. In these embodiments, annular protrusion 54 provides a flat surface in which to ultrasonically weld externally threaded cap 36. In some embodiments, flange 56 may provide an increased surface area and an attachment point for lever-locking cannula 16 (as shown in FIGS. 16-17 and described below).

In embodiments, recess 58 provides access to central fluid chamber 50, as illustrated in FIG. 8. Recess 58 allows slit septum 34 to be emplaced therein, so as to keep slit septum 34 secured and aligned with the primary fluid passageway 46. Recess 58 may include an internal beveled edge 60. Recess 58 may be configured such that at least a portion of the slit septum 34 is disposed within recess 58 when T-site connector 10 is assembled. Septum housing 42 may therefore present an external seat 62 and an internal seat 64. The external seat 62 is formed at least in part by annular protrusion 54 and flange 56. As best illustrated in FIG. 8, external seat 62 may be further formed by primary luer body 28. In some embodiments, external seat 62 provides a surface against which the externally threaded cap 36 may be secured. The internal seat 64 is formed at least in part by recess 58 and an inner recess 66 therein. As will be discussed in greater detail below, internal seat 64 provides a static point by which longitudinal compression of the slit septum 34 may occur.

Returning now to FIGS. 1-4, internally threaded collar 32 is secured to primary luer body 28 at the distal end 40 of T-site body 26. The internally threaded collar 32 includes an outer body 68 and a plurality of internal threads 70 for rotatably coupling internally threaded collar 32 to a vascular access device (VAD), such as catheter 12. The outer body 68 of internally threaded collar 32 may include one or more inner gripping protrusions 72 extending radially inwards to prevent internally threaded collar 32 from moving distally off of primary luer body 28. Similarly, primary luer body 28 may include one or more outer gripping protrusions 74 extending radially outward therefrom to retain internally threaded collar 32 thereon once emplaced. Internally threaded collar 32 is secured such the internally threaded collar 32 is free-spinning relative to primary luer body 28. Internally threaded collar 32 is configured to rotatably secure to a VAD, such as catheter 12. More specifically, the plurality of internal threads 70 may be rotated onto the catheter 12. In some embodiments and as will be discussed in greater detail below with respect to one or more external threads 234, the plurality of internal threads 70 may comprise two, three, four, or more threads. In some embodiments, the plurality of internal threads 70 may comprise three threads. Internally threaded collar 32 may include various anti-rotation features to keep internally threaded collar 32 secured to catheter 12.

As noted above, internally threaded collar 32 is removably coupled to catheter 12. As shown in FIG. 41, catheter 12 generally comprises an annular proximal base 76 with diametrically opposed connection tabs 78 for threaded connection to the internally threaded collar 32, an elongated barrel 80, and an elongated injection lumen 82 secured to a distal end of the elongated barrel 80. Embodiments of the invention may also be used with other catheter designs, as well as other components permanently or removably secured to T-site connector 10.

Before discussing slit septum 34 in detail, cannula 24 will be briefly described, as is illustrated in FIG. 19. Cannula 24 may be permanently or periodically inserted into the slit septum 34 so as to provide access to primary fluid passageway 46. As used herein, “cannula” may refer to a blunt cannula, needle, additional luers, or other similar medical device. Cannula 24 may be inserted into slit septum 34 at the proximal end 38 of T-site connector 10. In some embodiments, cannula 24 comprises a proximal annular base 84 and an externally ribbed barrel 86 terminating in an elongated injection lumen 88. In some embodiments, proximal annular base 84 may be provided with diametrically opposed connection tabs 78 (similar to the connection tabs 78 illustrated on catheter 12) configured for threaded connection with a standard luer lock fitting (as depicted in FIG. 19). In embodiments, cannula 24 may be a blunt cannula formed of a relatively rigid plastic or stainless steel and intended to provide needleless connection with primary fluid passageway 46. However, as noted above, embodiments presented herein may use both blunt cannulas and needles.

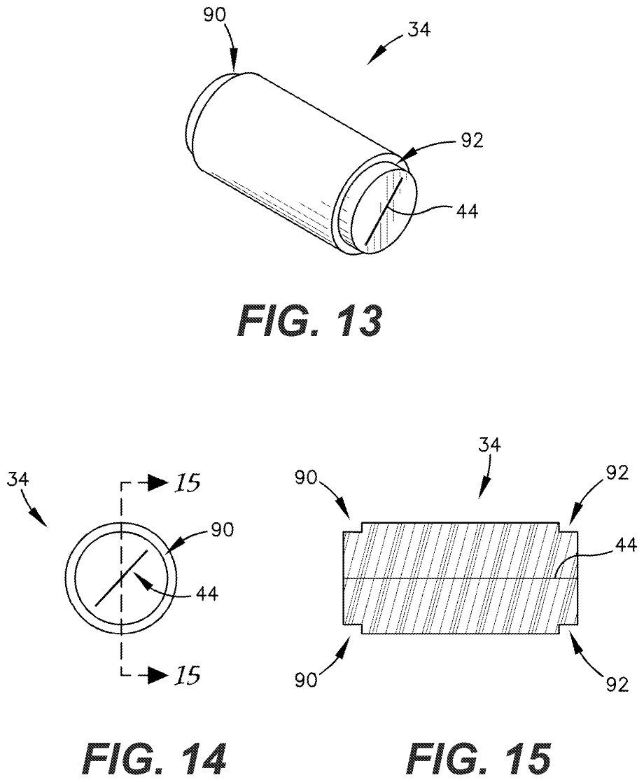

FIGS. 13-15 illustrate some embodiments of slit septum 34 having an axially formed slit 44 for receipt of cannula 24 (e.g., blunt cannula, needle, or other medical device) therethrough. In some embodiments, slit septum 34 presents a generally cylindrical shape, as best illustrated in FIG. 13. Slit septum 34 may include a first annular seat 90 and a second annular seat 92. First annular seat 90 and second annular seat 92 may comprise a recess, notch, channel, bevel, or other shape into at least a portion of the circumference of slit septum 34. In some embodiments, slit septum 34 is symmetrical (i.e., first annular seat 90 and second annular seat 92 are the same shape), such that either end of slit septum 34 may be placed against and into septum housing 42. This may allow for ease of manufacturing and assembly and may provide other benefits. In some embodiments, slit septum 34 is asymmetrical (i.e., first annular seat 90 and second annular seat 92 are not the same shape), such that a specific end, for example first annular seat 90, is configured to be placed against and into septum housing 42.

In some embodiments, slit septum 34 is formed of a synthetic isoprene with silica filler that may be free of natural rubber, latex, or mercaptobenzimidazol and has a hardness of between about 36 Shore A to about 41 Shore A. In some embodiments, the slit septum 34 is resilient and compressible. For example, in some embodiments slit septum 34 is compressible along both a longitudinal length and a transverse width. Slit septum 34 may be hermetically sealed within T-site connector 10 to significantly minimize or completely prevent the expulsion of fluids out of slit 44 in slit septum 34, that may otherwise occur upon removal of cannula 24 from slit 44.

In some embodiments, slit septum 34 is formed as a single, monolithic unit. The “pre-use position,” as referenced herein, is the position of slit septum 34 located within externally threaded cap 36 and septum housing 42 while externally threaded cap 36 is secured to annular protrusion 54 of septum housing 42, as illustrated in FIG. 4. In the pre-use position, slit septum 34 is under compression due to being in the externally threaded cap 36 and septum housing 42, but the slit septum 34 is not “in use” because the cannula 24, needle, or other medical device is not inserted into slit septum 34. Thus, the “in use” position is when the cannula 24, needle, or other medical device is inserted into slit septum 34. In contrast, a “rest” or “neutral” position is when slit septum 34 is not located in externally threaded cap 36 or septum housing 42, i.e., slit septum 34 is not under compression, as illustrated in FIG. 13.

Slit 44 is formed in slit septum 34 and extends along the entire longitudinal length of slit septum 34. In some embodiments, slit 44 comprises a single slit through slit septum 34. In other embodiments, slit 44 comprises a plurality of slits (not illustrated) formed in slit septum 34. For example, two slits may be formed in slit septum 34. In yet further embodiments, three or more slits may be formed in slit septum 34, such as tri-slit formed in a Y-shape. When slit septum 34 is in the pre-use or rest positions, slit 44 and slit septum 34 are in a closed position, i.e., fluid does not flow through slit 44. Upon insertion of cannula 24, or a needle or other medical device, into slit 44, slit septum 34 transitions to the “in use” position, thereby fluidly coupling cannula 24 and/or associated medical devices with T-site connector 10.

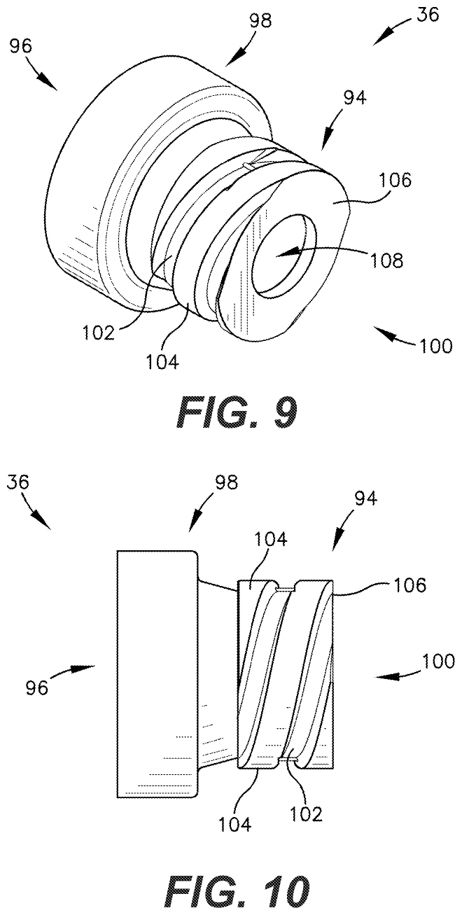

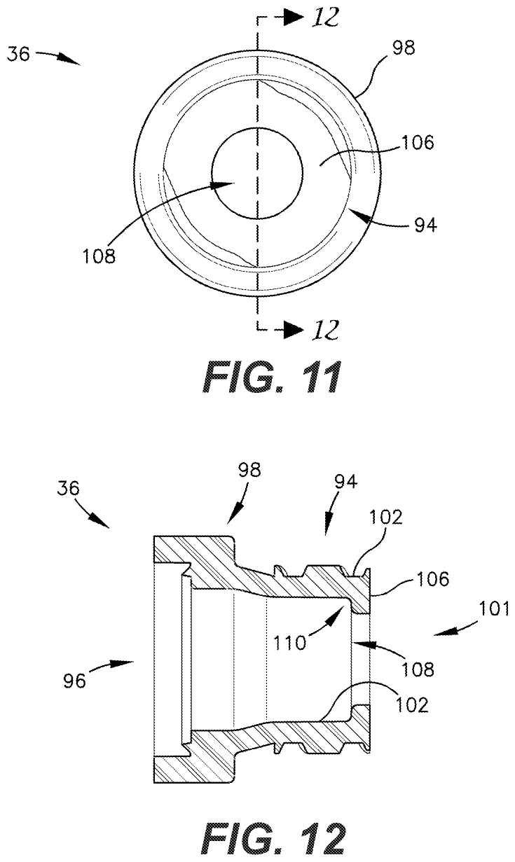

Slit septum 34 is secured to primary luer body 28 via externally threaded cap 36, as best illustrated in FIG. 4. Externally threaded cap 36 keeps slit septum 34 secured and aligned with septum housing 42 of T-site body 26. Externally threaded cap 36 presents an external-threaded segment 94, which is configured to receive a proximal connection device thereon (e.g., internally threaded collar 32, which may be internally threaded complementary to the external threading of the externally threaded cap 36), as best illustrated in FIGS. 9-12. In some embodiments and as discussed in greater detail below with respect to one or more external threads 234, external-threaded segment 94 may comprise two threads, three threads, four threads, or more. In some embodiments, external-threaded segment 94 may comprise three threads. In exemplary embodiments, the internal threading of the connection device (e.g., internally threaded collar 32) may be complementary to external-threaded segment 94.

In one example, the proximal connection device may be an antiseptic cap (such as an alcohol cap, not illustrated) configured to prevent or reduce infection by the transfer of cannula 24 into and out of slit septum 34. In another example, the proximal connection device may be a lever-locking cannula 16 (illustrated in FIGS. 16-17) or collar-locking cannula 300 (illustrated in FIGS. 38-40) that allows for quick attachment to and removal from primary luer body 28 (e.g., without the use of an internally threaded component). In another example, the proximal connection device may be a threaded-locking cannula 18 (illustrated in FIGS. 19-20) being associated with (or configured to receive) a syringe 19 at the proximal end for the injection of a medicament via T-site connector 10.

Externally threaded cap 36 presents a generally open-ended cylinder shape, as best illustrated in FIGS. 9-12. In some embodiments, externally threaded cap 36 is open on a distal end 96 to allow slit septum 34 to be emplaced therein, and to allow for externally threaded cap 36 to be mated to the complementarily shaped septum housing 42. Externally threaded cap 36 is open on a proximal end 100 to allow access to slit 44 of slit septum 34. Externally threaded cap 36 thus encases slit septum 34, keeps it secure against primary luer body 28, and allows cannula 24 to pierce slit 44 of slit septum 34.

In some embodiments, externally threaded cap 36 comprises a cap segment 98 and external-threaded segment 94, as best illustrated in FIG. 9. Cap segment 98 may be complementary to annular protrusion 54 such that cap segment 98 is configured to fit over annular protrusion 54. Cap segment 98 is configured to be secured to annular protrusion 54 of T-site body 26 so as to retain slit septum 34 within externally threaded cap 36 (e.g., see FIG. 4). As discussed above, this keeps slit septum 34 under compression, so as to prevent reflux of fluid through slit 44. In some embodiments, cap segment 98 is secured to annular protrusion 54 by ultrasonic welding, with slit septum 34 housed therein.

The external-threaded segment 94 presents an outer wall 102 and a one or more external threads 104, such as a helical ridge, protruding or otherwise extending radially therefrom. In some embodiments, and as discussed below with respect to one or more external threads 234, one or more external threads 104 comprise two, three, four, or more threads. In some embodiments, one or more external threads 104 comprise three threads. The one or more external threads 104 allow for a medical device (e.g., threaded-locking cannula 18) to be secured thereto, through rotation of internally threaded collar 32, primary luer body 28, or both (as illustrated in FIG. 19). Outer wall 102 presents a proximal face 106 that is flush with slit septum 34 (e.g., see FIG. 4). Proximal face 106 includes a septum opening 108 therein that is smaller in diameter than an outermost diameter of slit septum 34 but is the same size (or substantially the same size) as the proximal end of slit septum 34. As such, outer wall 102 presents a proximal seat 110 that is configured to fit with seat 90,92 of slit septum 34, so as to keep slit septum 34 secured and properly aligned.

In some embodiments, externally threaded cap 36 presents an inner proximal taper shape (which may be within cap segment 98 and/or external-threaded segment 94), as best illustrated in FIG. 12. In these embodiments, an inner diameter of externally threaded cap 36 is smaller near the proximal end 100 (e.g., away from the primary luer body 28) than it is at the distal end 96 (e.g., proximate to the primary luer body 28). Because, as discussed above, some embodiments of slit septum 34 present a cylindrical body, a tapered annular void 112 (as illustrated in FIG. 4) is presented between slit septum 34 and the inner proximal taper shape of cap segment 98. At least a portion of tapered annular void 112 is filled when slit septum 34 expands upon the insertion of cannula 24 therein. The tapered annular void 112 is configured such that it places an inward pressure on slit septum 34 while withdrawing cannula 24 (e.g., from the elastomeric expansion of the slit septum 34), and thereby prevents the unintended reflux of fluid, so as to reduce the risk of infection.

The taper shape of the tapered annular void 112 will now be discussed in more detail. As illustrated in FIG. 4, the proximal end of tapered annular void 112 may present a first radial distance (not labeled) and the distal end of tapered annular void 112 may present a second radial distance. The first and second radial distances each present an axial width of the gap between slit septum 34 and septum housing 42. In some embodiments, such as those depicted in FIG. 4, the proximal axial width may be half of the distal axial width. In some embodiments, the distal axial width is greater than the proximal axial width. The tapered annular void 112 may present a tapered transition therebetween, such that the width of tapered annular void 112 is greater at the distal end compared to the proximal end.

As mentioned above, tapered annular void 112 is configured to receive portions of slit septum 34 upon insertion of cannula 24 into slit 44, due to the elastomeric expansion of slit septum 34. Because of the respective axial widths, elastomeric expansion takes place with the motion of the cannula 24 being inserted into the slit septum 34. As such, the tapered annular void 112 is filled in a proximal to distal direction as the cannula 24 is inserted.

In some embodiments in which cannula 24 is twisted during attachment, the screwing motion of inserting the cannula 24, when compared to traditional methods of straight insertion, allows for additional torque to be applied. The additional torque may overcome additional pressure placed on slit septum 34 due to the bounds of septum housing 42. The screwing action may also straighten cannula 24 and prevent piercing the walls of slit septum 34.

Various exemplary proximal connection devices will now be discussed. The discussed proximal connection devices may be connected to primary luer body 28 and/or tube bond stem 30. It should be appreciated that any of numerous types of medical devices may be secured to primary luer body 28 via slit septum 34 and/or tube bond stem 30. Four exemplary structures are depicted in FIGS. 16-19 and FIG. 38. These exemplary proximal connection devices are lever-locking cannula 16, threaded-locking cannula 18, tube set 20, and collar-locking cannula 300. Some embodiments of the invention are directed to an injection site system, including T-site connector 10 (such as described above) and one or more of the lever-locking cannula 16, the threaded-locking cannula 18, the tube set 20, and the collar-locking cannula 300. The injection site system may also include a VAD, such as catheter 12 shown in FIG. 41.

An example of the lever-locking cannula 16 is illustrated in FIGS. 16-17. More specifically, FIGS. 16-17 illustrate lever-locking cannula 16 prior to (FIG. 16) and following (FIG. 17) attachment to T-site connector 10, as viewed from above (e.g., in the direction of the secondary fluid passageway 48). As such, FIGS. 16-17 are best viewed together for the following description. In some embodiments, lever-locking cannula 16 includes a first pivot lock 114, a second pivot lock 116, a base 118, and a cannula 121. First pivot lock 114 and second pivot lock 116 are each rigidly secured to base 118. Each of the first pivot lock 114 and the second pivot lock 116 present a proximal lever 120 and a distal clip 122.

The lever-locking cannula 16, as illustrated in FIGS. 16-17, is utilized by the medical professional applying slight inward pressure to each proximal lever 120 (such as with a thumb and forefinger). The inward pressure moves distal clips 122 outward. The distal clips 122 moving outward allows for the distal clips 122 to be installed around flange 56, concurrently with the cannula 121 inserting into slit 44 of slit septum 34. The medical professional then releases the inward pressure on proximal levers 120 such that distal clips 122 are relaxed to a default, locked position. In some embodiments, base 118 of lever-locking cannula 16 may be attached to a syringe (such as syringe 19 illustrated in FIG. 19) or other container of a medicament for injection into the patient. It should be noted that, as illustrated in FIGS. 16-17, the external-threaded segment 94 of externally threaded cap 36 (depicted in FIG. 12) is essentially bypassed in this configuration, to allow for quick attachment and detachment.

Some embodiments of the threaded-locking cannula 18 are illustrated in FIGS. 19-21. More specifically, FIG. 19 illustrates threaded-locking cannula 18 installed between T-site connector 10 and syringe 19. FIG. 20 and FIG. 21 each depict the threaded-locking cannula 18 alone. The threaded-locking cannula 18 comprises a base 124, a cannula 24, and an internally threaded collar segment 126. Threaded-locking cannula 18 may provide a more secure and more permanent attachment than lever-locking cannula 16. In some embodiments, internally threaded collar segment 126 is free-spinning relative to cannula 24 and base 124. Base 124 may be configured to interface with syringe 19 or other proximal medical devices 128.

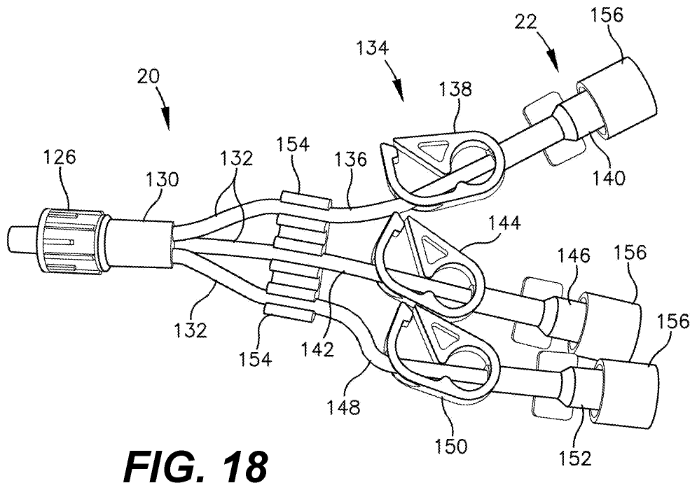

Some embodiments of tube set 20 are illustrated in FIG. 18. Tube set 20 is configured to provide a plurality of access ports 22. Tube set 20 is configured to attach to any one of lever-locking cannula 16, threaded-locking cannula 18, locking cannula 280, or collar-locking cannula 300 by an internally threaded collar segment 126, located at central hub 130. Thus, tube set 20 may be fluidly coupled to T-site connector 10/200 by means of lever-locking cannula 16, threaded-locking cannula 18, locking cannula 280, or collar-locking cannula 300. Tube set 20 may also be configured to be secured to the tube bond stem 30 via an internally threaded collar segment 126, pure mechanical compression via a taper, or other structure. In some embodiments, tube set 20 comprises a central hub 130, a plurality of lines 132 leading to central hub 130, a plurality of flow locks 134, and a plurality of access ports 22.

As in the example shown in FIG. 18, tube set 20 may comprise a first line 136 associated with a first flow lock 138 and a first access port 140, a second line 142 associated with a second flow lock 144 and a second access port 146, and a third line 148 associated with a third flow lock 150 and a third access port 152. Tube set 20 may further include an alignment clip 154 for keeping each of the plurality of lines 132 in a spaced-apart orientation. In some embodiments, tube set 20 may include a port cover 156 associated with each access port 22. Port cover 156 may be an antiseptic cap. The port cover 156 of these embodiments includes a porous inner material for retaining an antiseptic liquid therein, such that a portion of the antiseptic liquid is emplaced on access port 22 upon emplacing port cover 156 thereon.

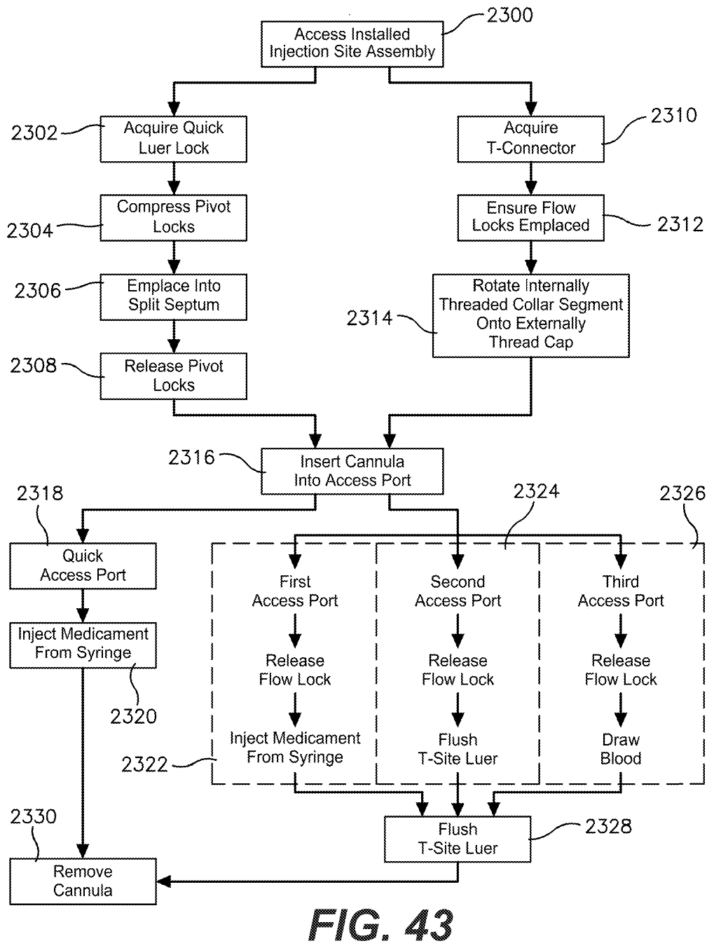

Each access port 22 and/or flow lock 134 may be of a certain color or have some other distinguishing mark or characteristic. This is because each access port 22 may be used for a single purpose (as discussed below with respect to FIG. 43). For example, the first flow lock 138 may be blue to signify injections using the first access port 140, the second flow lock 144 may be white signifying flushing using the second access port 146, and the third flow lock 150 may be red signifying blood draw using the third access port 152. It should be appreciated that the above is merely an example and that various embodiments of tube set 20 may use other color schemes and/or markers, have more or fewer lines 132, etc.

In some embodiments, cannula 24 presents a length configured to terminate at the central fluid chamber 50. This reduces the blood and other fluids that come in contact with cannula 24. As cannula 24 is removed from slit septum 34, the blood or other fluids may be deposited along slit septum 34 or in other proximate locations, which can lead to blood stream infections and other complications. In some embodiments, cannula 24 presents a length that is slightly less than a length of slit septum 34 such that cannula 24 will terminate while still slightly within slit septum 34 to further reduce the contact between blood and other fluids with cannula 24. It should be appreciated that while FIG. 19 illustrates cannula 24 fully piercing into central fluid chamber 50 (labeled in FIG. 8), in other embodiments cannula 24 may insert to central fluid chamber 50 and/or short of central fluid chamber 50. In some embodiments, slit septum 34 presents a longitudinal length based upon a standard length of cannula 24 for this purpose.

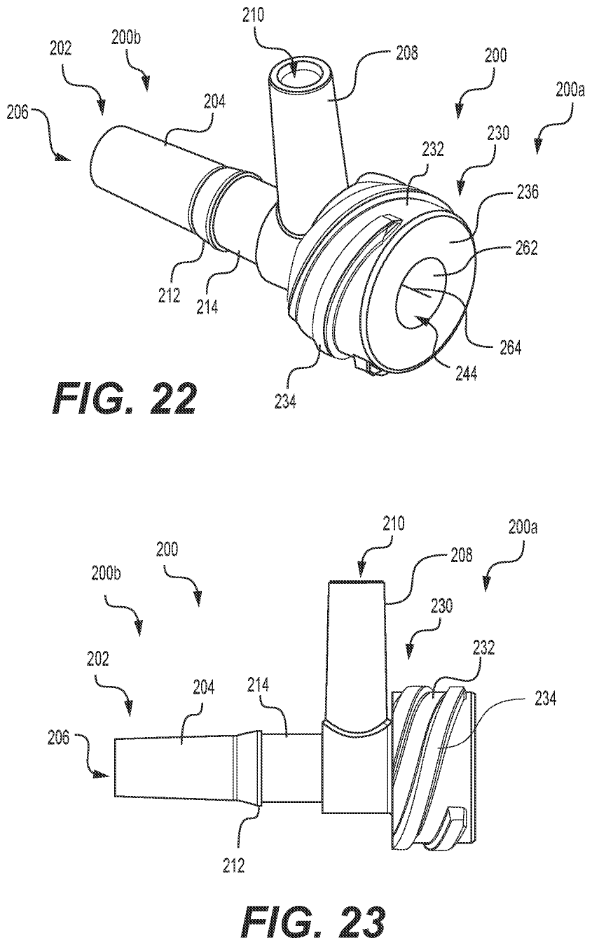

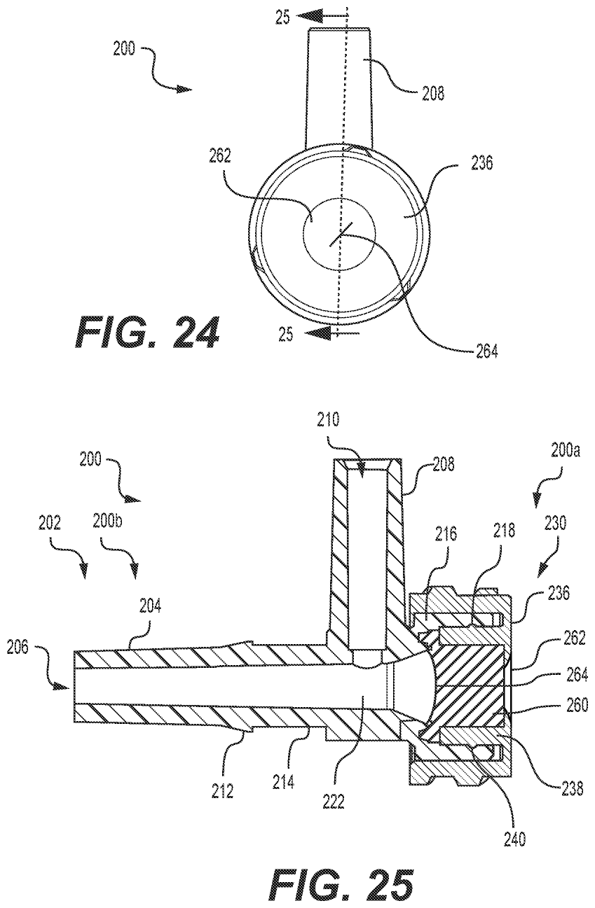

FIGS. 22-25 depict some embodiments of T-site connector 200. FIG. 22 depicts some embodiments of a proximal perspective view of T-site connector 200. FIG. 23 depicts some embodiments of a side view of T-site connector 200. FIG. 24 depicts some embodiments of an end view of T-site connector 200. FIG. 25 depicts a vertical cross-sectional view of T-site connector 200. As such, FIGS. 22-25 are best viewed together for the following description.

As depicted, T-site connector 200 may have some similar components, connectivity, and structure as portions of T-site connector 10. As will be appreciated and described below, in some embodiments, T-site connector 200 and T-site connector 10 have exchangeable portions. For example, internally threaded collar 32, which may be secured to primary luer body 28, as described above, may similarly be secured to primary luer body 204 (not shown). Similarly, in some embodiments T-site connector 10 and T-site connector 200 may share similar structural features. For example, similar to the one or more inner gripping protrusions disposed on primary luer body 28 of T-site connector 10, T-site connector 200 may include one or more gripping protrusions 212 disposed on primary luer body 204 so as to capture internally threaded collar 32. In some embodiments, T-site connector 10 and T-site connector 200 may include differing components or structures. For example, externally threaded cap 36 of T-site connector 10 may comprise a different outer diameter than externally threaded cap 230 of T-site connector 200. These examples should not be construed as limiting the similarities or differences between T-site connector 10 and T-site connector 200. As mentioned previously, any number of combinations of parts may or may not be shared between T-site connector 10 and T-site connector 200.

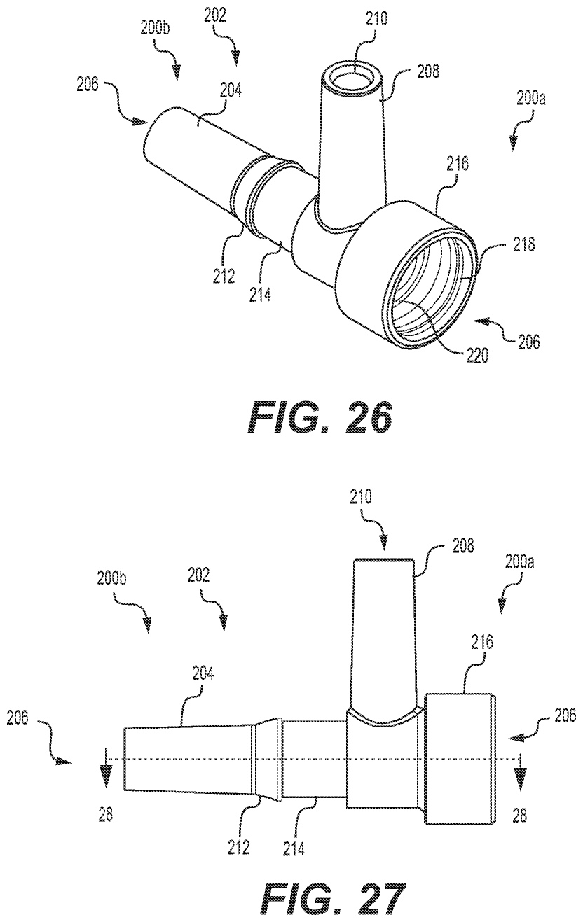

T-site connector 200 includes a proximal end 200a and a distal end 200ib. In some embodiments, T-site connector 200 includes a T-site body 202 extending from the proximal end 200a to the distal end 200b. As shown in FIGS. 26-28, T-site body 202 may include primary luer body 204 extending distally and defining a primary fluid passageway 206 therein and along the longitudinal axis. As discussed above with reference to primary fluid passageway 46, primary fluid passageway 206 may act as the main fluid passageway through T-site connector 200.

As briefly mentioned above, in some embodiments primary luer body 204 may include one or more gripping protrusions 212 extending radially outward therefrom. In some embodiments, primary luer body 204 may include capturing portion 214 located proximally to one or more gripping protrusions 212. Capturing portion 214 may comprise a smaller radius than one or more gripping protrusions 212. Similar to the description above, one or more gripping protrusions 212 and capturing portion 214 may retain and/or secure an internally threaded collar (e.g., internally threaded collar 32) thereto. Also similar to the above description, one or more gripping protrusions 212 may prevent distal movement of the internally threaded collar (e.g., internally threaded collar 32) past a certain point. However, the internally threaded collar (e.g., internally threaded collar 32) may not be attached or adhered to primary luer body 204 such that the internally threaded collar may freely rotate around primary luer body 204 allowing primary luer body 204 to securely attach to a VAD (e.g., catheter 12) by way of the internally threaded collar.

In some embodiments, T-site connector 200 includes tube bond stem 208 defining a secondary fluid passageway 210 therethrough. Similar to descriptions above, secondary fluid passageway 210 intersects primary fluid passageway 206 at a central fluid chamber 222, such that fluids administered via tube bond stem 208 pass through secondary fluid passageway 210 and to primary fluid passageway 206. Accordingly, medicaments may be administered via tube bond stem 208.

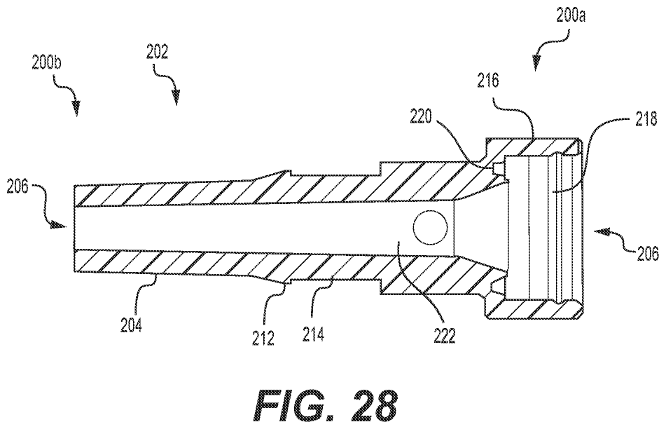

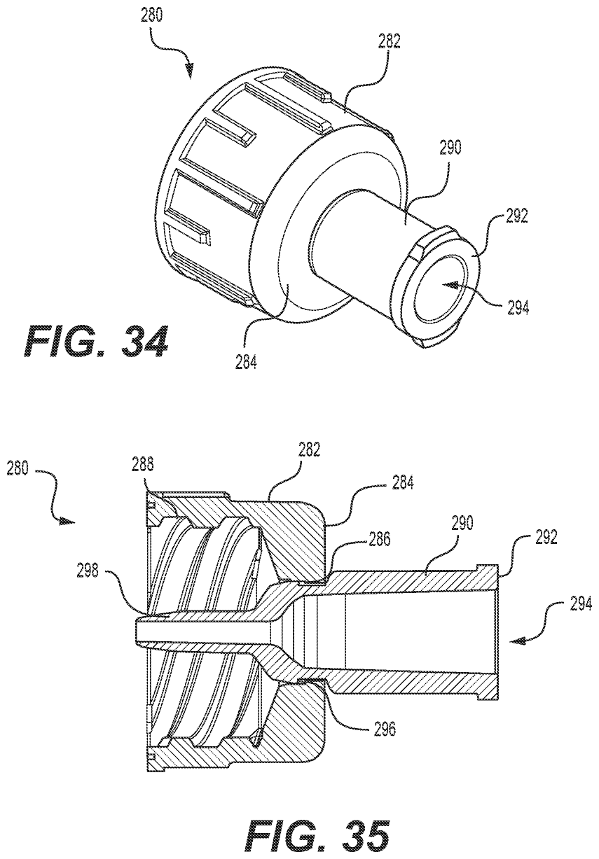

In some embodiments, T-site body 202 includes septum housing 216 extending longitudinally towards proximal end 200a. In some embodiments, septum housing 216 may be configured to house a portion, or all of septum 260. Furthermore, septum housing 216 may engage a portion of septum 260 so as to prevent or preclude distal movement of septum 260 when attaching T-site connector 200 to a cannula (e.g., cannula 290 depicted in FIGS. 34-37). For example, septum housing 216 may include internal seat 220 (e.g., see FIGS. 26 and 28) configured to engage housing protrusion 270 extending distally from septum 260 (e.g., see FIG. 33).

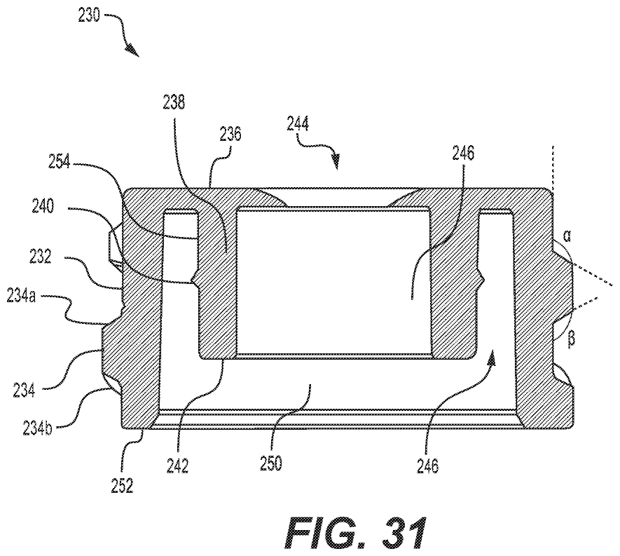

As depicted in FIG. 25, in some embodiments septum housing 216 may be configured to attach to externally threaded cap 230 and thereby secure septum 260 therein. For example, in some embodiments septum housing 216 may include an internal recess 218 forming a recessed segment within septum housing 216. Further, as will be discussed in greater detail below, externally threaded cap 230 may include an inner flange 238 extending distally from proximal face 236. As shown in FIGS. 30-31, inner flange 238 may define a pocket 248 which may be configured to receive septum housing 216 therein, as shown in FIG. 25. In some embodiments, inner flange 238 includes an annular protrusion 240 extending radially therefrom and into pocket 248. As such, internal recess 218 may be configured to receive annular protrusion 240 therein upon connection of externally threaded cap 230 to T-site body 202, thereby securing externally threaded cap 230 to T-site body 202. The reception site of annular protrusion 240 within internal recess 218 may be further secured to strengthen the connection of externally threaded cap 230 to T-site body 202. For example, annular protrusion 240 may be ultrasonically welded to internal recess 218. In another example, annular protrusion 240 may be chemically adhered to internal recess 218. In yet other embodiments, the geometry of annular protrusion 240 and internal recess 218 may be such that reception of annular protrusion 240 into internal recess 218 causes a friction fit between externally threaded cap 230 and T-site body 202, thereby securing externally threaded cap 230 to T-site body 202.

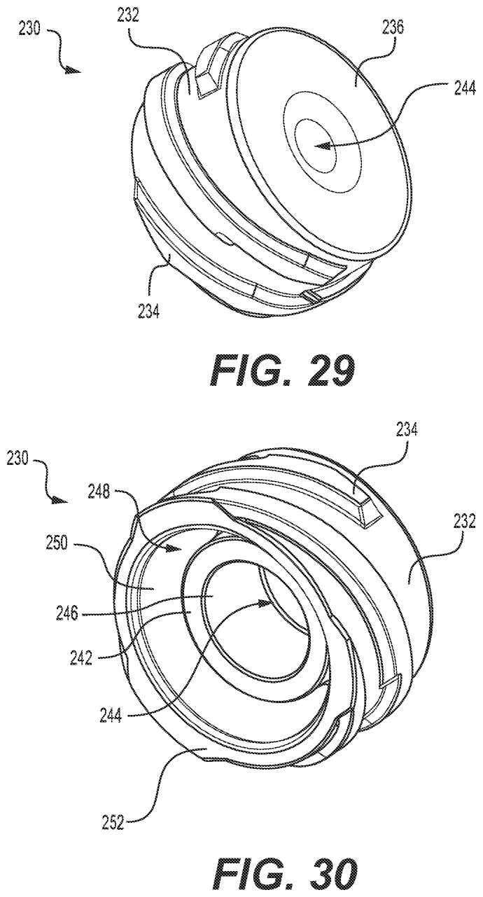

In some embodiments, portions of septum 260 are accessible while septum 260 is secured within septum housing 216 via externally threaded cap 230. For example, as depicted in FIGS. 22 and 24-25, septum 260 may include a swabbable face 262 that is accessible through bore 244 disposed on externally threaded cap 230 and through proximal face 236 (see FIG. 29). Accordingly, swabbable face 262 may be swabbable while T-site connector 200 is assembled, thereby helping prevent infection by removing and/or destroying microbes, viruses, or other harmful organisms or compounds located on swabbable face 262. Further, septum 260 includes slit 264 that is accessible when septum 260 is secured within septum housing 216. As such, slit 264 may receive a cannula (e.g., cannula 290) therethrough when T-site connector 200 is assembled.

Similar to T-site connector 10, various proximal medical devices 128 may be connected to T-site connector 200 at proximal end 200a and/or tube bond stem 208. Three exemplary structures are shown in FIGS. 16-21 and 34-35. These exemplary proximal connection devices include lever-locking cannula 16, threaded-locking cannula 18, tube set 20, and locking cannula 280.

Externally threaded cap 230 will now be discussed in greater detail. FIG. 29 depicts a proximal perspective view of externally threaded cap 230, in some embodiments. FIG. 30 depicts a distal perspective view of externally threaded cap 230, in some embodiments. FIG. 31 illustrates a vertical cross-sectional view of externally threaded cap 230, in some embodiments. Accordingly, FIGS. 29-31 are best viewed together for the following description of externally threaded cap 230. As described above, externally threaded cap 230 includes proximal face 236 having bore 244 extending therethrough. In some embodiments, proximal face 236 may be substantially perpendicular to the longitudinal axis (i.e., flat). In some embodiments, proximal face 236 may comprise an indented, tapered shape, such that bore 244 is directed distally towards swabbable face 262.

In some embodiments externally threaded cap 230 includes external wall 232 extending distally from proximal face 236. External wall 232 includes one or more external threads 234 projecting radially outwardly therefrom and configured to fasten to an internally threaded collar (e.g., internally threaded collar segment 282 depicted in FIGS. 34-37). As mentioned previously, as externally threaded cap 230 is securely fastened to t-site body 202, rotation of internally threaded collar segment 282 thereabout may securely fasten internally threaded collar segment 282 to externally threaded cap 230 via one or more external threads 234. In some embodiments, one or more external threads 234 may include a stop or other shape that aid in securing internally threaded collar segment 282.

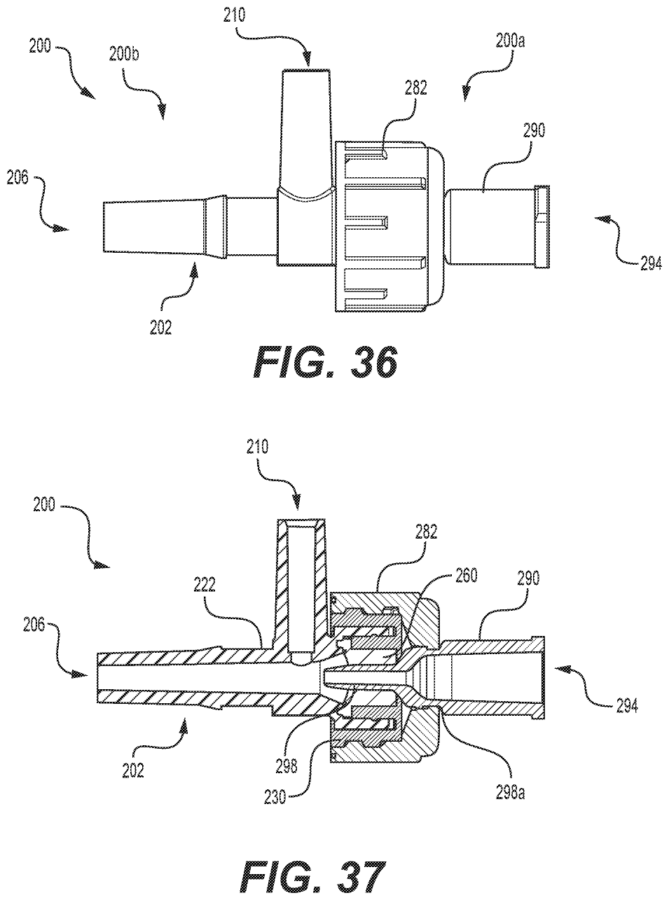

In some embodiments, the number and structure of external threads 234 may be adjusted such that different degrees of rotation of internally threaded collar segment 282 may be required to fully fasten internally threaded collar segment 282 to externally threaded cap 230. For example, in some embodiments one or more external threads 234 may comprise two threads. In these embodiments, rotation of internally threaded collar segment 282 between about 330 degrees to about 370 degrees may fully fasten internally threaded collar segment 282 to externally threaded cap 230 (e.g., upon full reception of externally threaded cap 230 within internally threaded collar segment 282, such as depicted in FIGS. 36-37). In some embodiments, one or more external threads 234 may comprise three threads. In these embodiments, rotation of internally threaded collar segment 282 between about 210 degrees to about 250 degrees may fully fasten internally threaded collar segment 282 to externally threaded cap 230. In some embodiments, one or more external threads 234 may comprise four threads. In these embodiments, rotation of internally threaded collar segment 282 between about 150 degrees to about 190 degrees may fully fasten internally threaded collar segment 282 to externally threaded cap 230. In each of the aforementioned embodiments, internally threaded collar segment 282 may comprise internal threads 288 configured to receive each of the one or more external threads 234. For example, in embodiments in which one or more external threads 234 comprises two threads, internally threaded collar segment 282 may comprise two internal threads 288. In another example, in embodiments in which one or more external threads 234 comprises three threads, internally threaded collar segment 282 may comprise three internal threads 288. In another example, in embodiments in which one or more external threads 234 comprises four threads, internally threaded collar segment 282 may comprises four internal threads 288.

Further, as depicted in FIG. 31, one or more external threads 234 may comprise an angle α and an angle β that define the angles by which proximal wall 234a and distal wall 234b of one or more external threads 234 extend from external wall 232, respectively. In some embodiments, angle α and angle β may further define the degrees of rotation needed to securely and fully fasten internally threaded collar segment 282 to externally threaded cap 230. For example, in some embodiments, angle α may be between about 90 degrees to about 130 degrees. In some embodiments, angle α may be between about 100 degrees to about 120 degrees. In some embodiments, angle α may be about 110 degrees. Further in some embodiments, angle β may be between about 100 degrees to about 140 degrees. In some embodiments, angle β may be between about 110 degrees to about 130 degrees. In some embodiments, angle β may be about 120 degrees.

As is apparent from the above description, an increase in the number of one or more external threads 234 and internal threads 288 leads to a decrease in the degrees of rotation necessary to secure internally threaded collar segment 282 to externally threaded cap 230. Similarly, an increase in angle α and/or a decrease in angle β leads to a decrease in the degrees of rotation necessary to secure internally threaded collar segment 282 to externally threaded cap 230. However, there may be limitations to the number of one or more external threads 234 and internal threads 288 that will still allow for rotational connection of internally threaded collar segment 282 to externally threaded cap 230. For example, increasing the number of one or more external threads 234 and internal threads 288 may decrease the likelihood of internal threads 288 receiving one or more external threads 234 therein upon rotation. Accordingly, a certain number of threads and angle of angle α and angle β may be used to optimize the operator's fastening of externally threaded cap 230 to internally threaded collar segment 282. In exemplary embodiments, one or more external threads 234 may comprise three threads, angle α may be about 110 degrees, and angle β may be about 120 degrees. It should be understood that the above is merely illustrative, and any number of one or more external threads 234, and degree of angle α and angle β may be chosen to optimize the ability of an operator to rotationally secure internally threaded collar segment 282 to externally threaded cap 230. It is to be further understood that any number of threaded portions on T-site connector 10 or T-site connector 200 may similarly be configured to enhance the capability of a user to secure components to T-site connector 10 or T-site connector 200. For example, in some embodiments externally threaded cap 36 may comprise three threads 104. In another example, internally threaded collar 32 may comprise internal threads 70 configured to receive and secure to a device having three external threads. In another example, internally threaded collar segment 126 may comprise internal threads configured to receive and secure to a device having three external threads.

As briefly described above and depicted in FIGS. 30 and 31, externally threaded cap 230 may include inner flange 238 extending distally from proximal face 236. In some embodiments, inner flange 238 includes septum barrier wall 246 defining an inner portion. In some embodiments such as those depicted in FIG. 25, septum barrier wall 246 may be configured to abut septum wall 266 substantially or completely when T-site connector 200 is assembled.

In some embodiments, inner flange 238 terminates distally at axial wall 242. As will be discussed in greater detail below, axial wall 242 may be configured to abut septum flange 268 when T-site connector 200 is assembled, thereby helping to maintain positioning of septum 260 within T-site connector 200.

As described above, inner flange 238 may further include an outer wall 254. In some embodiments, outer wall 254 connects to septum barrier wall 246 via axial wall 242. Further, as illustrated in FIG. 31, outer wall 254 comprises a wider diameter than septum barrier wall 246 and therefore defines the width of axial wall 242. As mentioned above, outer wall 254 includes annular protrusion 240 extending outwardly therefrom and configured to be received by a portion of T-site body 202 (e.g., internal recess 218).

As briefly described above, outer flange 252 may define septum housing reception wall 250 located internally. In embodiments, septum housing reception wall 250 defines a greater diameter than outer wall 254. As such, outer wall 254 and septum housing reception wall 250 define pocket 248 therebetween, whereby pocket 248 is configured to receive some or all of septum housing 216 therein (e.g., see FIG. 25). As such, when septum housing 216 is received within pocket 248, annular protrusion 240 extends into internal recess 218, thereby securing externally threaded cap 230 to T-site body 202.

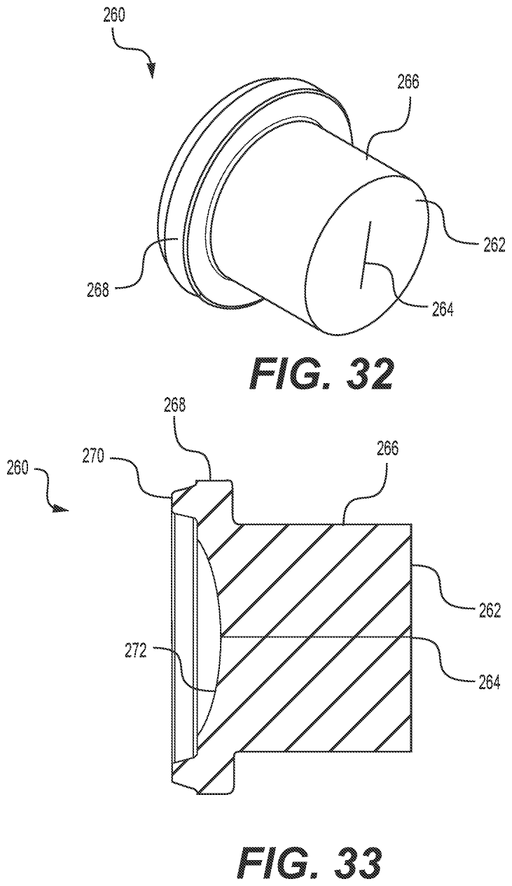

FIG. 32 depicts a proximal perspective view of septum 260, in some embodiments. FIG. 33 illustrates a vertical cross-sectional view of septum 260, in some embodiments. As such, FIGS. 32 and 33 are best viewed together for the following description.

As briefly mentioned above, septum 260 includes swabbable face 262 containing slit 264 therethrough. In embodiments, slit 264 extends longitudinally through the entirety of septum 260. Similar to slit 44, slit 264 is configured to receive a cannula therethrough. Septum 260 also includes septum wall 266 extending distally from swabbable face 262. When T-site connector 200 is assembled, some of all of septum wall 266 may abut septum barrier wall 246 of externally threaded cap 230. Such abutment of septum wall 266 against septum barrier wall 246 may aid in maintaining septum 260 in a closed, or pre-use position. Further, abutment of septum wall 266 against septum barrier wall 246 may aid in maintaining a seal of slit 264 against a cannula tip (e.g., cannula tip 298 of cannula 290) when extending therethrough (i.e., when septum 260 is in the “in use” position).

In some embodiments, septum 260 includes a septum flange 268 extending radially outward from septum wall 266 at the distal end of septum 260. As illustrated in FIG. 25, septum flange 268 may abut axial wall 242 of externally threaded cap 230 when T-site connector 200 is assembled. In some embodiments, axial wall 242 may exert a distal force on septum flange 268, thereby helping to maintain the position of septum 260 within T-site connector 200.

In some embodiments, septum flange 268 includes housing protrusion 270 extending distally therefrom. As illustrated in FIG. 25, housing protrusion 270 may be configured to extend into internal recess 218 of T-site body 202 when T-site connector 200 is assembled. Such a configuration may aid in maintaining the position of septum 260 within T-site connector 200. Further, in some embodiments abutment of axial wall 242 and septum flange 268 in combination with housing protrusion 270 being received within internal recess 218 may aid in preventing distal movement of septum 260 when distal pressure is exerted on septum 260 while inserting a cannula into slit 264.

Septum 260 includes a distal wall 272. When T-site connector 200 is assembled, distal wall 272 leads to central fluid chamber 222. Distal wall 272 may comprise a concave (e.g., as illustrated in FIG. 33), flat, or convex shape. The shape of distal wall 272 may prevent or preclude reflux of fluids through slit 264 upon or during removal of the cannula. For example, during removal of a cannula from slit 264, negative pressure may occur thereby biasing fluid in the proximal direction (i.e., reflux). In some embodiments, a concave shape of distal wall 272 may aid in preventing fluid reflux through slit 264 when removing a cannula.

FIG. 34 depicts a proximal perspective view of a locking cannula 280 in some embodiments. FIG. 35 depicts a vertical cross-sectional view of locking cannula 280, in some embodiments. As such, FIGS. 34 and 35 are best viewed together for the following description.

Similar to threaded-locking cannula 18 described above, locking cannula 280 may attach to T-site connector 200 at proximal end 200a, thereby allowing connection of syringe 19 or other proximal medical device 128 to T-site connector 200. Locking cannula 280 includes an internally threaded collar segment 282 and a cannula 290. Internally threaded collar segment 282 includes a proximal wall 284 having a hole 284a that receives a portion of cannula 290 therethrough. Internally threaded collar segment 282 may include a retention feature to maintain attachment of internally threaded collar segment 282 to cannula 290 when received therein. For example, in some embodiments internally threaded collar segment 282 may include an inner ridge 286 disposed within hole 284a and configured to engage a portion of cannula 290 (e.g., protruding ridge 296) when cannula 290 is received therein.

Internally threaded collar segment 282 includes one or more internal threads 288 configured to receive one or more external threads 234 of externally threaded cap 230 therein. Similar to embodiments described above in relation to one or more external threads 234, one or more internal threads 288 may comprise one, two, three, four, or more internal threads. In embodiments, the number of external threads 234 and internal threads 288 is the same. In some embodiments, one or more external threads 234 comprises three threads and one or more internal threads 288 comprises three threads.

Cannula 290 includes a proximal annular base 292 similar to proximal annular base 84. In embodiments, proximal annular base 292 allows for secure attachment of cannula 290 to a syringe (e.g., syringe 19) or other proximal medical device 128. Further, cannula 290 includes cannula fluid path 294 therethrough, extending from proximal annular base 292 to cannula tip 298. As will be discussed below, cannula fluid path 294 allows for fluid flow between syringe 19 or other proximal medical device 128 and T-site connector 200 when locking cannula 280 is attached to T-site connector 200.

Referring now to FIGS. 36 and 37, T-site connector 200 is depicted in some embodiments with locking cannula 280 attached at proximal end 200a. As mentioned above, internally threaded collar segment 282 rotatably attaches to externally threaded cap 230 by reception of one or more external threads 234 within internal threads 288. In some embodiments, as internally threaded collar segment 282 rotatably attaches to externally threaded cap 230, cannula tip 298 inserts through bore 244 and into slit 264. As cannula tip 298 inserts into slit 264, outward force presses septum wall 266 against septum barrier wall 246. This pressure causes slit 264 to maintain a seal against cannula tip 298, thereby not allowing any fluid to flow therebetween. Additionally, insertion of cannula tip 298 into slit 264 exerts a distal force on septum 260. In some embodiments, positioning of septum flange 268 and housing protrusion 270 of septum 260 prevent distal movement of septum 260. For example, abutment of septum flange 268 against axial wall 242 along with insertion of housing protrusion 270 into internal seat 220 helps maintain positioning of septum 260 and thereby prevent distal translation of septum 260 while cannula tip 298 inserts in the distal direction through slit 264.

In some embodiments, septum 260 is formed of a synthetic isoprene with silica filler that may be free of natural rubber, latex, or mercaptobenzimidazol and has a hardness of between about 36 Shore A to about 41 Shore A. In some embodiments, the septum 260 is resilient and compressible. Septum 260 may be compressible along both the longitudinal length and a transverse width. Septum 260 may be hermetically sealed within the T-site connector 200 to significantly minimize or completely prevent the expulsion of fluids out of slit 264 in septum 260, that may otherwise occur upon removal of cannula tip 298 from slit 264.

The length of cannula tip 298 may be such that once internally threaded collar segment 282 is fully rotated and secured onto externally threaded cap 230, cannula tip 298 may extend distally through slit 264 and past distal wall 272, placing septum 260 in the “in use” position. Furthermore, the configuration and sizing of slit 264, septum 260, and cannula tip 298 is such that a friction fit forms between cannula tip 298 and septum 260, thereby preventing fluid movement therebetween. Thus, once locking cannula 280 is fully attached to externally threaded cap 230, a complete fluid path is formed from cannula fluid path 294 to central fluid chamber 222 and primary fluid passageway 206. Such a configuration allows for the exchange of fluids and/or medicaments between syringe 19, or other proximal medical device 128, and the patient.

In some embodiments, such as is depicted in FIG. 37, a cannula tip protrusion 298a may extend radially outward from cannula tip 298, whereby the cannula tip protrusion 298a comprises a diameter larger than the diameter of bore 244. As such, distal movement of cannula tip 298 through bore 244 may be prevented once cannula tip protrusion 298a abuts the perimeter of bore 244 (see FIG. 37). Further, as described above, the position of cannula 290 within hole 284a of internally threaded collar segment 282 is maintained due to retention features (e.g., inner ridge 286 and protruding ridge 296) thereby preventing proximal movement of cannula 290 from through hole 284a. Accordingly, as cannula tip protrusion 298a of cannula 290 begins to contact the perimeter of bore 244, cannula 290 and cannula tip 298 are stabilized by pressure between inner ridge 286 and protruding ridge 296, thereby maintaining cannula 290 and cannula fluid path 294 straight with regards to the longitudinal axis of primary fluid passageway 206.

As discussed above, additional proximal connection devices may be connected to T-site connector 10 and/or T-site connector 200. Some embodiments of a proximal connection device, collar-locking cannula 300, are depicted in FIGS. 38-40. FIGS. 38-40 are best viewed together for the following description. Similar to lever-locking cannula 16, collar-locking cannula 300 may be used to quickly attach and detach from T-site connector 10/200. For example, a single application of a medicament may be performed by quickly attaching collar-locking cannula 300 by translating and rotating collar-locking cannula 300 such that tube bond stem 30/208 is placed into retention aperture 310 (see description below), injecting the medicament, and detaching collar-locking cannula 300. Similar to the description regarding FIG. 37, the attachment of collar-locking cannula 300 to t-site connector 10/200 forms a friction fit between cannula 290 (e.g., cannula tip 298) and septum 260. Collar-locking cannula 300 includes proximal end 302 and collar 304. As discussed below, collar 304 is configured to receive and retain tube bond stem 30/208 to transiently maintain connection of collar-locking cannula 300 to T-site connector 10/200.

In some embodiments, collar 304 includes retaining wall 306 having collar extension 308 extending therefrom and defining retention aperture 310. In some embodiments, collar 304 includes guiding wall 312 configured to guide tube bond stem 30 as tube bond stem 30 is received within collar-locking cannula 300. The termination end of collar extension 308 in combination with guiding wall 312 form reception aperture 314. The retention process of tube bond stem 30/208 within collar-locking cannula 300 will now be described.

As mentioned above, collar-locking cannula 300 may be configured to quickly attach and detach from T-site connector 10 and/or T-site connector 200. While some descriptions herein may reference connection of collar-locking cannula 300 to T-site connector 10 or T-site connector 200 for clarity purposes, it is to be understood that collar-locking cannula 300 may be configured to attach to T-site connector 10 and T-site connector 200 in similar ways. When connecting collar-locking cannula 300 to T-site connector 10/200, tube bond stem 30/208 is received first within reception aperture 314 as collar-locking cannula 300 is translated towards proximal end 38/200a of T-site connector 10/200. Furthermore, as tube bond stem 30/208 is received within reception aperture 314, guiding wall 312 may guide tube bond stem 30/208 within reception aperture 314 and prevent rotation of collar-locking cannula 300 during initial translation of collar-locking cannula 300. During translation, externally threaded cap 36/230 is received within the hollowed-out middle of collar 304. In these embodiments, similar to the description above with relation to lever-locking cannula 16, external-threaded segment 94 of externally threaded cap 36 or one or more external threads 234 of externally threaded cap 230 are bypassed in this configuration. Said another way, in some embodiments in which collar-locking cannula 300 is used, externally threaded cap 36 and externally threaded cap 230 lack external-threaded segment 94 and one or more external threads 234, respectively.

Once tube bond stem 30/208 reaches the most proximal portion of reception aperture 314, tube bond stem 30/208 is then to be retained within retention aperture 310. To retain tube bond stem 30/208 within retention aperture 310, the user rotates collar-locking cannula 300 in a first direction with respect to T-site connector 10/200. For example, the user may grip proximal end 302 and rotate collar-locking cannula 300 in the first direction while holding T-site connector 10/200 stationary. Such rotation moves tube bond stem 30/208 from reception aperture 314 into retention aperture 310. Once tube bond stem 30/208 is rotated into retention aperture 310, collar-locking cannula 300 is prevented from moving distally, thereby maintaining insertion of cannula tip 298 within slit septum 34 or septum 260.

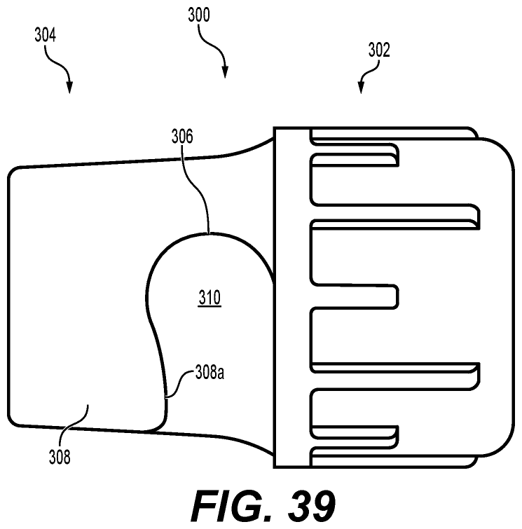

In some embodiments, collar extension 308 may include collar edge 308a, best illustrated in FIG. 39. Collar edge 308a may extend distally within retention aperture 310, thereby aiding in the capture of tube bond stem 30/208 within retention aperture 310. For example, collar edge 308a may prevent rotation of collar-locking cannula 300 unless collar-locking cannula 300 is translated proximally in relation to T-site connector 10/200. In other words, collar edge 308a may prevent accidental disconnect of collar-locking cannula 300 from T-site connector 10/200. Therefore, to disconnect collar-locking cannula 300 from T-site connector 10/200, a user may translate collar-locking cannula 300 proximally compared to T-site connector 10/200. During or following proximal translation of collar-locking cannula 300, the user may rotate collar-locking cannula 300 in a second direction, opposite the first direction, to place tube bond stem 30/208 within reception aperture 314. The user may then disconnect collar-locking cannula 300 from T-site connector 10/200 by translating collar-locking cannula 300 distally and moving tube bond stem 30/208 along guiding wall 312 and out of reception aperture 314.

As stated above with regards to lever-locking cannula 16, collar-locking cannula 300 may provide the advantage of rapid connection and disconnection of the proximal connection device from the T-site connector 10/200.

Various methods of using the above-described components will now be discussed. Some embodiments of the invention are directed to using at least one of the above-described components. Various methods may be utilized by medical professionals. Examples of medical professionals can include doctors, anesthesiologists, physician assistants, nurses, nurse practitioners, certified nursing assistants, medics, phlebotomists, emergency medical technicians, veterinarians, veterinary assistants, and others. Some embodiments of the methods may be performed by the patient, by a family member of the patient, by a friend of the patient, or other person.

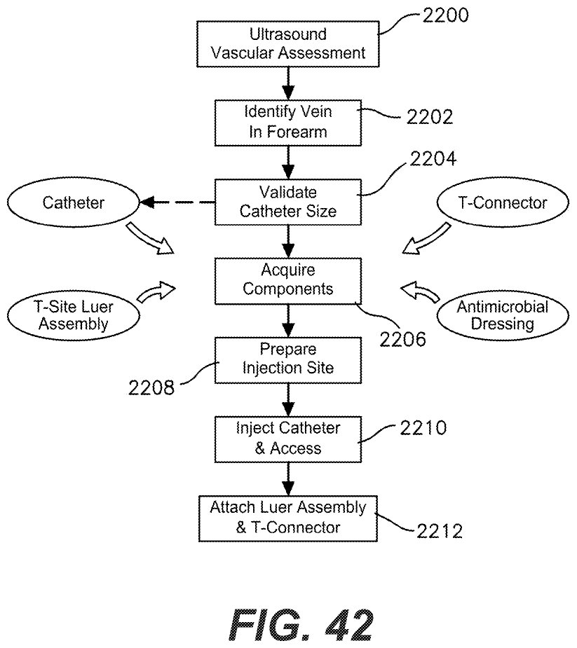

Exemplary methods of some embodiments disclosed herein are shown in FIGS. 42 and 43. Generally, FIG. 42 shows a method of installing and connecting T-site connector 10/200 to the patient. Generally, FIG. 43 shows a method of utilizing and upkeep of the installed T-site connector 10/200. For clarity purposes, the below descriptions of steps illustrated in FIGS. 42 and 39 are described as using T-site connector 10 and/or T-site connector 200 and their associated components. It is to be understood that, as described above, components associated with T-site connector 10 may be installed and/or otherwise used with T-site connector 200, and vice versa. It should be appreciated that the various steps illustrated in both FIG. 42 and FIG. 43 may be performed in any order and are not necessarily self-contained. For example, some steps of FIG. 43 may be performed before all of the steps of FIG. 42 are completed.

Turning to FIG. 42, a method of installing and fluidly connecting T-site connector 10/200 to the patient is shown and described. In some embodiments of the invention, a single T-site connector 10/200 is used for a single injection for the entirety of the patient's stay at the medical facility. This single injection is then used for all or most procedures requiring the injection of a medicament and the withdrawal of blood. Limiting to a single injection site may be advantageous to reduce the pain and discomfort caused to the patient (because each intravenous and arterial injection is physically painful for the patient). Limiting to a single injection site may also be advantageous because it reduces the workload required to be performed by the medical personnel (because each intravenous and arterial injection is time consuming and requires skill to perform). Limiting to a single injection site may also be advantageous because it reduces the risk of blood reflux (because each intravenous administration has an independent risk of blood reflux). Reduction of blood reflux reduces the risk of blood clotting, occlusion of T-site connector 10/200, and infection.

In a step 2200, the medical professional performs, on a forearm of a patient, an ultrasound vascular assessment. In an example of step 2200, the medical professional performs an ultrasound vascular assessment on a patient to locate a vein for insertion of catheter 12, or other VAD, into. The ultrasound vascular assessment identifies the size, location, and orientation of a target vein in the forearm of the patient. In prior art systems, vascular assessments have been used to identify and diagnose medical conditions, such as blood clots and other blockages, by evaluating the flow of blood through the veins and arteries. Because, as discussed above, T-site connector 10/200 uses a single injection access point for the duration of a patient stay at a medical establishment, the ultrasound vascular assessment may be used to ensure an accurate injection the first time. The ultrasound vascular assessment also allows the utilization of a vein in forearm (e.g., in the forearm below the elbow). While veins at the inner elbow are easier to access, they are more painful and prone to infection.

In a step 2202, the medical professional identifies, potentially based on the ultrasound vascular assessment performed at step 2200, a target vein in the forearm of the patient. In an example of step 2202, the medical profession identifies and labels a vein that may be capable of receiving and retaining catheter 12, or other VAD, for the duration of the stay of the patient, or an otherwise extended period of time (i.e., longer than a single injection period). As discussed above, the ultrasound vascular assessment may be used to determine which vein to utilize, ensure that it is accessible, and determine if the other forearm of the patient should be used instead. The identified vein may be marked or otherwise noted.

In a step 2204, the medical professional determines, potentially based on the ultrasound vascular assessment performed at step 2200, a size of catheter that is appropriate for the target vein. In an example of step 2204, the size of the catheter 12 may be determined based upon the approximate diameter of the vein, the orientation of the vein, the length of the vein that is proximate to the skin, and/or other considerations. Unlike prior art systems in which the size of catheter would be chosen based upon a standardization for a type of patient, embodiments disclosed herein allow for the size of catheter 12 to be chosen specifically for the patient.

In some embodiments, the size of the catheter 12 may be determined or suggested by a computer program that is associated with the ultrasound vascular assessment. For example, a computer program running or otherwise receiving data from the ultrasound may identify the vein and measure the width of the vein as detected by the ultrasound. The determined width of the vein (which corresponds to the diameter) may be used by the computer program to determine or calculate the correct size of catheter. It should be appreciated that the above-discussed computer program may be associated with a computerized method of performing the discussed steps or may be stored on a non-transitory computer readable storage medium such that execution of the computer program by a processor performs the discussed steps.

In a step 2206, the medical professional obtains the components needed. In an example of step 2206, the medical profession obtains a catheter 12 having the size determined in step 2204. The medical profession may also obtain any of T-site connector 10/200, lever-locking cannula 16, threaded-locking cannula 18, locking cannula 280, collar-locking cannula 300, and/or tube set 20. In some embodiments, the medical professional may also obtain the proximal medical device 128, such as a syringe 19, intravenous drip bags, blood draw vials, and the like. Other equipment for the injection may also be obtained, such as reconstitution solution (such as sterile water or specific diluent), alcohol swabs, cotton wool, the dressing to cover the injection site, and the like.

In a step 2208, the medical professional prepares an injection site on the forearm of the patient. In an example of step 2208, the medical profession prepares the injection site by applying a disinfectant to the site (such as an alcohol-based solution) and allowing the disinfectant to dry.

In a step 2210, the medical professional injects the catheter into the target vein of the forearm of the patient. In an example of step 2210, the medical professional injects catheter 12 into the forearm of the patient.

In a step 2212, the medical professional attaches a T-site connector to the catheter. In an example of step 2212, the medical professional attaches T-site connector 10/200 to catheter 12. Note that in some embodiments, T-site connector 10/200 may be attached to catheter 12 prior to the catheter 12 being injected into the target vein (i.e., step 2212 is performed prior to step 2210). The T-site connector 10/200 is then ready for usage as the single access point for the injection of medicaments to and withdrawal of blood from the patient's bloodstream. The medical professional may also place an antiseptic cap, such as port cover 156, over the externally threaded cap 36/230 to prevent infection.

Turning to FIG. 43, a method of utilizing T-site connector 10/200 is shown and described. The method of utilization may be interspersed with steps of upkeep and maintenance of the injection site. Some examples of upkeep and maintenance include: 1) periodic assessment of catheter functionality; 2) monitoring of adhesion of the dressing that covers the catheter injection site; 3) periodically changing the dressing; 4) periodic flushing of the T-site connector 10/200; and 5) periodic changing of the antiseptic cap and/or the application of a disinfectant to the other components.

FIG. 43 illustrates some steps for the usage of two different proximal connection devices. Usage of the lever-locking cannula 16 is shown and described generally on the left side of FIG. 43. Usage of the tube set 20 is shown and described generally on the right side of FIG. 43. Steps that are common to both proximal connection devices are shown in the center of FIG. 43. It should also be appreciated that usage of the threaded-locking cannula 18 may be described generally on the right side on the top half of FIG. 43 (steps 2300, 2310, 2312, 2314, and 2316) and the left side on the bottom half of FIG. 43 (steps 2318, 2320, and 2330).

In a step 2300, the medical practitioner accesses the installed T-site connector (e.g., T-site connector 10/200). In an example of step 2300, the T-site connector 10/200 may have been installed, such as described above in regard to FIG. 42 immediately prior to the below discussed steps. In another example of step 2300, the T-site connector 10/200 may have been installed at an earlier time. In another example of step 2300, an antiseptic cap (e.g., port cover 156) is removed so as to access the septum (e.g., septum 260) and externally threaded cap (e.g., externally threaded cap 230).

First, connection of a lever-locking cannula will be discussed. In a step 2302, the medical practitioner acquires the lever-locking cannula (e.g., lever-locking cannula 16). In an example of step 2302, the lever-locking cannula may be (previously or subsequently) secured to a syringe (e.g., syringe 19), a vacutainer or other blood container, or other proximal medical device 128. In some embodiments, the lever-locking cannula 16 is used for single use, rapid injections. The lever-locking cannula 16 may be used to quickly attach and detach from the T-site connector 10/200 (or to and from other proximal connection devices). For example, a single application of a medicament may be performed by quickly attaching lever-locking cannula 16, injecting the medicament, and detaching lever-locking cannula 16.

In a step 2304, the medical practitioner compresses the pivot locks located on the lever-locking cannula. In an example of step 2304, the pivot locks (e.g., first pivot lock 114 and second pivot lock 116) on lever-locking cannula 16 are compressed, such as between a thumb and forefinger. The lever-locking cannula 16 is utilized by the medical professional applying slight inward pressure to each proximal lever 120. The inward pressure thus moves the distal clips 122 outward.

In a step 2306, the medical professional inserts the cannula of the lever-locking cannula into the septum of the T-site connector. In an example of step 2306, the distal clips 122 move outward, thereby allowing for the distal clips 122 to be installed around a flange (e.g., flange 56) located on the proximal end of the T-site connector, concurrently with the cannula piercing the septum (e.g., through slit 264 of septum 260).

In a step 2308, the medical professional releases the pivot locks. In an example of step 2308, the medical professional releases the inward pressure on the proximal lever 120 such that distal clips 122 are relaxed to a default, locked position.

Next, connection of a tube set 20 or threaded-locking cannula 18 will be discussed. In a step 2310, the medical professional acquires the tube set and/or threaded-locking cannula. In an example of step 2310, the medical profession acquires tube set 20 and/or threaded-locking cannula 18. The appropriate proximal connection devices may have one or more proximal medical devices 128 (such as a syringe 19, vacutainer, or the like) already attached thereto.

In a step 2312, the medical professional ensures that flow locks of the tube set are emplaced. In an example of step 2312, the medical professional emplaces flow locks 134 on tube set 20. In some embodiments, the tube set 20 includes a flow lock 134 on each line 132 (as discussed above). Such a configuration may prevent reflux out of the unused line 132 upon injection to or withdrawal from the other lines 132.

In a step 2314, the medical professional attaches the proximal connection devices to the proximal end of the T-site connector (e.g., to the externally threaded cap, to the antiseptic cap, or other component), or to the tube bond stem, depending on the application. In an example of step 2314, the medical professional may attach to the proximal end 200a of the T-site connector 200, a distal end of locking cannula 280. In another example of step 2314, the medical professional may attach to the proximal end 200a of T-site connector 200, a distal end of tube set 20 including the first access port 140, the second access port 146, and the third access port 152.

As described above, in embodiments in which threaded-locking cannula 18 or locking cannula 280 is attached to externally threaded cap 36/230, the degree of rotation necessary for fully securing threaded-locking cannula 18 or locking cannula 280 to externally threaded cap 36/230 may be decreased. For example, in some embodiments one or more external threads 104/234 may comprise an increased number of threads to decrease the degree of rotation required to secure threaded-locking cannula 18 or locking cannula 280 to externally threaded cap 36/230. For example, in some embodiments one or more external threads 104/234 may comprise three external threads and thereby require a rotation of between about 220 degrees to about 240 degrees of internally threaded collar 126/282 to fully secure threaded-locking cannula 18 or locking cannula 280 to externally threaded cap 104/234. Such a decrease in required rotation may make it easier for a user of T-site connector 10/200 to connect threaded-locking cannula 18 or locking cannula 280. For example, in practice a user may use two fingers to rotatably attach threaded-locking cannula 18 or locking cannula 280 to externally threaded cap 36/230. Accordingly, a rotation of 300 degrees or greater may make it difficult for the user to complete such a rotation without releasing and reengaging internally threaded collar 126/282. Further, increasing the number of external threads may increase the likelihood of engagement between internally threaded collar 126/282 and externally threaded cap 36/230. For example, less rotation may be required to cause insertion of the one or more external threads 104/234 into the internal threads disposed on internally threaded collar 126/282.

In some embodiments, the medical professional may connect the proximal connection device to the tube bond stem 30/208 via an intermediary tube segment 158, as illustrated in FIG. 44. The intermediary tube segment 158 includes a flexible tube 160 and a female luer lock 162. The flexible tube 160 is configured to fit into the tube bond stem 30/208 (see FIGS. 5 and 22). The medical professional places and presses a bottom end 164 of the flexible tube 160 into the tube bond stem 30/208. In some embodiments, friction holds the bottom end 164 of the flexible tube 160 inside of the tube bond stem 30/208. In some embodiments, the bottom end 164 of the flexible tube 160 may be solvently bonded within tube bond stem 30/208. In some embodiments, the bottom end 164 of the flexible tube 160 may be adhesively bonded within tube bond stem 30/208. Such connection of intermediary tube segment 158 to tube bond stem 30/208 may allow for the flow of liquids and medication through secondary fluid passageway 48/210.

The female luer lock 162 is configured to receive and connect to a male luer found on any needleless connector, syringe, or IV administration set. As mentioned above, by connecting female luer lock 162 to a syringe, for example, medicaments and/or liquids may be exchanged between the syringe and patient via secondary fluid passageway 48/210. The intermediary tube segment 158 may further comprise a flow lock 134, which may be similar or the same as flow locks 134 described in relation to FIG. 18. The flow lock 134 controls the overall flow between tube bond stem 30 and the proximal connection device attached thereto via the intermediary tube segment 158.

In a step 2316, a cannula is inserted into an access port. In an example of step 2316, cannula 24 is inserted into access port 22. In another example of step 2316, a proximal medical device 128, such as syringe 19, is connected to threaded-locking cannula 18 or locking cannula 280. The cannula, such as a blunt cannula, a needle, or other similarly shaped device, is used to access the interior of the proximal connection devices (which is in fluid communication with the primary fluid passageway 46/206 of the T-site body 26/202 and the patient blood stream). It should be noted that in some embodiments, this step is performed prior to the above-discussed steps, based upon the application.

In a step 2318, the medical professional prepares the access port. In an example of step 2318, the medical professional prepares the access port by removing port cover 156 if applicable and/or applying an antiseptic wipe.

In a step 2320, the medical professional injects a medicament into the T-site connector (and by extension, into the blood stream of the patient). In an example of step 2320, the medical professional injects a medicament from syringe 19, through T-site connector 10/200 and into the patient. This is best illustrated in FIG. 19 which shows the syringe 19 connected to the T-site body 26 through the threaded-locking cannula 18. In this configuration a plunger of the syringe 19 is depressed so as to force the medicament therein into the patient's blood stream.

Steps 2322-26 illustrate three exemplary uses for tube set 20. It should be appreciated that the tube set 20 may have more or fewer access ports 22, and the access ports 22 may be used for additional or different functions. Similarly, as discussed above, the access ports 22 may have a distinctive color or marking designating the specific access port 22 for one or more specific purpose.

In a step 2322, the medical professional utilizes a first access port of the tube set. In an example of step 2322, the medical professional utilizes first access port 140 of tube set 20. In some embodiments, first access port 140 may be designated for the injection of medicaments. In this example of step 2322, the medical professional inserts in the first access port 140, a medicament cannula (e.g., cannula 24 associated with a medicament, such as syringe 19 or an intravenous drip bag). The medical professional removes a first flow lock 138 from the first line 136 associated with the first access port 140. This can be done by unclipping the first flow lock 138 so as to allow fluid to flow therethrough. The medical professional subsequently injects, into the patient, a medicament from the cannula 24 via the first access port 140.

In a step 2324, the medical professional utilizes the second access port of the tube set. In an example of step 2324, the medical professional utilizes the second access port 146 of tube set 20. In this example of step 2324, the medical professional inserts cannula 24 into the second access port 146. The medical professional removes the second flow lock 144 from the second line 142 associated with the second access port 146. This can be done by unclipping the second flow lock 144 so as to allow fluid to flow therethrough. The medical professional then flushes the T-site connector 10/200 by supplying a flushing liquid therein from the cannula 24 via the second access port 146. In some embodiments, second access port 146 may be designated for flushing the T-site body 26/202. Flushing may be performed to remove all residual medicament (such as an antibiotic) before a blood draw. Such flushing may be necessary, as having antibiotics in the blood draw may corrupt the sample. In another example, flushing may be performed periodically to reduce the risk of infection growing within the T-site body 26/202.

In a step 2326, the medical professional utilizes the third access port of the tube set. In an example of step 2326, the medical profession inserts, in the third access port 152 (or in the slit septum 34), a cannula 24 configured for drawing blood. The medical professional then removes the third flow lock 150 from the third line 148 associated with the third access port 152. As discussed above, this can be done by unclipping the third flow lock 150. The medical professional then draws blood from the patient into the cannula 24 via the third access port 152, and into an attached vacutainer or other proximal medical device 128.

In some embodiments, the third access port 152 may be designated for blood draw. In other embodiments, slit septum 34 or septum 260 is designated for blood draw. In prior art systems, each blood draw was performed by piercing an artery for each blood draw procedure. This method is painful to the patient, time consuming, and prone to infection, as discussed above. Further, it is typical in prior art systems for the medical professional to draw far more blood than is needed for a given medical test. For example, it is standard to fill an entire 10 mL vacutainer, even when only 2 mL is needed for a test. The remainder is wasted. This can be problematic, for example, because a draw of 50 mL can drop hemoglobin by 18%. Because the blood draw procedure is performed independently, it has been standard to fill an entire vacutainer regardless of need. One goal of the current invention, therefore, may be to reduce the amount of blood drawn to more specifically match the test to be performed. Because the procedure is simplified from prior art systems, the medical professional may draw only as much blood as is needed.

In some embodiments, following the blood draw, the blood within one or more lines 132 may be returned to the patient by flushing. This can include applying a flushing cannula (e.g., cannula 24) to the first access port 140 and/or the third access port 152. Returning the blood to the patient can be important for some patients, especially those with low hemoglobin levels and other medical issues. Returning the blood to the patient allows for minimal wastage of blood. Returning the blood to the patient also reduces the risk of blood stream infection because the blood is not allowed to remain within the T-site connector 10/200 for an extended period of time, where an infection may develop.

It should also be appreciated that the discussed steps may be performed independently of the other discussed steps. For example, the flushing discussed in step 2324 may be performed before the blood draw discussed in step 2326, and the injection of the medicament described in step 2322 may be performed independently.

In a step 2328, the medical professional emplaces the flow lock that was removed to allow for access to the access port. In an example of step 2328, the medical professional emplaces the flow lock 134 that was removed to allow for access to the access port 22. In this example of step 2328, the flow lock 134 is emplaced by clipping the respective flow lock 134 so as to prevent fluid to flow therethrough. This prevents reflux of blood and/or the medicament from escaping when the cannula 24 is withdrawn, because the back pressure (e.g., from the blood stream of the patient) is cut off by the flow lock 134.

In a step 2330, a common step to any proximal connection device, the medical professional removes the cannula from the T-site connector. In an example of step 2330, the medical professional removes cannula 24 from T-site connector 10/200. The cannula 24 is then typically discarded, such as in a sharp disposal box. The medical professional may then apply an antiseptic cap to the access port 22, wipe the access port 22 with an antiseptic, and/or perform other sanitary functions so as to prevent or reduce the chance of infection.

Features described above as well as those claimed below may be combined in various ways without departing from the scope hereof. The following examples illustrate some possible, non-limiting combinations:
(A1) A T-site connector configured to fluidly attach a proximal medical device with a blood vessel of a patient, the T-site connector including: a T-site body including: a septum housing at a proximal end having a primary luer body extending distally therefrom and defining a primary fluid passageway; and a tube bond stem extending axially from the primary luer body and defining a secondary fluid passageway therein, the primary fluid passageway and the secondary fluid passageway being fluidly coupled at a central fluid chamber; an externally threaded cap including: a proximal face disposed at a proximal end of the externally threaded cap, the proximal face including a bore therethrough, the bore defining a first diameter; an external wall extending distally from the proximal face and having one or more external threads disposed circumferentially therearound; and an inner flange extending distally from the proximal face and within the external wall, the inner flange defining a second diameter being larger than the first diameter; and a septum housed within the inner flange and the septum housing, the septum including a slit extending longitudinally therethrough, the slit being accessible through the bore.
(A2) For the T-site connector denoted as (A1), the external wall and the inner flange of the externally threaded cap define a pocket therebetween, the pocket receiving the septum housing therein.
(A3) For the T-site connector denoted as (A1) or (A2), the septum housing includes an internal recess and the inner flange of the externally threaded cap includes an annular protrusion extending outwardly therefrom, the internal recess receiving the annular protrusion upon reception of the septum housing within the pocket.
(A4) For the T-site connector denoted as any of (A1) through (A3), the externally threaded cap further includes an axial wall disposed at a distalmost portion of the inner flange, the axial wall abutting a flange of the septum, the flange extending radially outward from a distal end of the septum.
(A5) For the T-site connector denoted as any of (A1) through (A4), the septum includes a housing protrusion extending distally from the flange, and the septum housing includes an internal seat receiving the housing protrusion.
(A6) For the T-site connector denoted as any of (A1) through (A5), the septum further includes a distal wall leading to the central fluid chamber, the distal wall including a concave shape.
(A7) For the T-site connector denoted as any of (A1) through (A6), the one or more external threads includes three external threads.
(B1) An injection apparatus, including: a T-site connector and a locking cannula, the T-site connector including: a T-site body, a septum, and an externally threaded cap, the T-site body including a septum housing fluidly coupled to a primary luer body and a tube bond stem; the septum including a swabbable face having a slit therethrough and extending to a distal wall; and the externally threaded cap secured to the septum housing of the T-site body and including a proximal face and an external wall extending distally therefrom, the external wall including one or more external threads disposed thereon, the septum is retained between the externally threaded cap and the septum housing; and the locking cannula including: a cannula having a proximal annular base connected to a cannula tip and defining a cannula fluid path therein; and an internally threaded collar segment receiving the cannula tip therein and being rotatable therearound, the internally threaded collar segment includes one or more internal threads configured to rotatably attach to the one or more external threads of the externally threaded cap.
(B2) For the injection apparatus denoted as (B1), the one or more external threads of the externally threaded cap includes three external threads and the one or more internal threads of the internally threaded collar segment includes three internal threads.
(B3) For the injection apparatus denoted as (B1) or (B2), a rotation of between 220 degrees to 240 degrees secures the internally threaded collar segment to the externally threaded cap.
(B4) For the injection apparatus denoted as any of (B1) through (B3), the cannula tip extends through the slit of the septum upon the rotation of the internally threaded collar segment, thereby fluidly connecting the locking cannula to the T-site body.
(B5) For the injection apparatus denoted as any of (B1) through (B4), the externally threaded cap further includes an inner flange extending distally from the proximal face, the inner flange defining a septum barrier wall abutting a septum wall of the septum.
(B6) For the injection apparatus denoted as any of (B1) through (B5), the inner flange further defines an outer wall forming a pocket between the external wall and the outer wall, the outer wall including an annular protrusion extending outwardly into the pocket.
(B7) For the injection apparatus denoted as any of (B1) through (B6), the septum housing includes an internal recess configured to receive the annular protrusion.
(C1) A system for controlling flow of liquids to and from a patient, including: a T-site connector including: a T-site body extending in a longitudinal direction between a proximal end and a distal end, the proximal end includes a septum housing and the distal end includes a primary luer body having a tube bond stem disposed therebetween and extending axially in relation to the longitudinal direction; a septum disposed within the septum housing, the septum including a slit therethrough; an externally threaded cap disposed at the proximal end, the externally threaded cap including: a proximal face having a bore therethrough; and an external wall extending distally from the proximal face and having one or more external threads disposed therearound, the externally threaded cap is secured to the septum housing, thereby retaining the septum within the septum housing; and a first internally threaded collar disposed at the primary luer body and being rotatable therearound; a locking cannula including: a cannula; and a second internally threaded collar receiving a portion of the cannula therethrough, the second internally threaded collar including one or more internal threads configured to receive the one or more external threads of the externally threaded cap.
(C2) For the system denoted as (C1), a rotation of between 220 degrees to 240 degrees of the second internally threaded collar secures the second internally threaded collar to the externally threaded cap.
(C3) For the system denoted as (C1) or (C2), the one or more internal threads of the second internally threaded collar includes three internal threads and the one or more external threads of the externally threaded cap includes three external threads.
(C4) For the system denoted as any of (C1) through (C3), the septum includes a flange extending radially outwards at a distal end of the septum, the flange being compressed between the externally threaded cap and the septum housing.
(C5) For the system denoted as any of (C1) through (C4), the flange includes a housing protrusion extending distally therefrom, further the septum housing includes an internal seat receiving the housing protrusion therein.
(C6) For the system denoted as any of (C1) through (C5), the cannula includes a cannula tip at a distalmost end and a cannula tip protrusion disposed proximally to the cannula tip, the cannula tip protrusion configured to abut a perimeter of the bore upon securing the second internally threaded collar to the externally threaded cap.

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. A T-site connector configured to fluidly attach a proximal medical device with a blood vessel of a patient, the T-site connector comprising:
a T-site body comprising:
a septum housing at a proximal end having a primary luer body extending distally therefrom and defining a primary fluid passageway; and
a tube bond stem extending axially from the primary luer body and defining a secondary fluid passageway therein, the primary fluid passageway and the secondary fluid passageway being fluidly coupled at a central fluid chamber;
an externally threaded cap comprising:
a proximal face disposed at a proximal end of the externally threaded cap, the proximal face comprising a bore therethrough, the bore defining a first diameter;
an external wall extending distally from the proximal face and having one or more external threads disposed circumferentially therearound; and
an inner flange extending distally from the proximal face and within the external wall, the inner flange defining a second diameter being larger than the first diameter; and
a septum housed within the inner flange and the septum housing, the septum comprising a slit extending longitudinally therethrough, the slit being accessible through the bore.

2. The T-site connector of claim 1, wherein the external wall and the inner flange of the externally threaded cap define a pocket therebetween, the pocket receiving the septum housing therein.

3. The T-site connector of claim 2, wherein the septum housing comprises an internal recess and the inner flange of the externally threaded cap comprises an annular protrusion extending outwardly therefrom, the internal recess receiving the annular protrusion upon reception of the septum housing within the pocket.

4. The T-site connector of claim 3, wherein the externally threaded cap further comprises an axial wall disposed at a distalmost portion of the inner flange, the axial wall abutting a flange of the septum, the flange extending radially outward from a distal end of the septum.

5. The T-site connector of claim 4, wherein the septum includes a housing protrusion extending distally from the flange, and wherein the septum housing comprises an internal seat receiving the housing protrusion.

6. The T-site connector of claim 5, wherein the septum further comprises a distal wall leading to the central fluid chamber, the distal wall comprising a concave shape.

7. The T-site connector of claim 6, wherein the one or more external threads comprises three external threads.

8. An injection apparatus, comprising:
a T-site connector and a locking cannula,
the T-site connector comprising:
a T-site body, a septum, and an externally threaded cap,
the T-site body comprising a septum housing fluidly coupled to a primary luer body and a tube bond stem;
the septum comprising a swabbable face having a slit therethrough and extending to a distal wall; and
the externally threaded cap secured to the septum housing of the T-site body and comprising a proximal face and an external wall extending distally therefrom, the external wall comprising one or more external threads disposed thereon,
wherein the septum is retained between the externally threaded cap and the septum housing; and
the locking cannula comprising:
a cannula having a proximal annular base connected to a cannula tip and defining a cannula fluid path therein; and
an internally threaded collar segment receiving the cannula tip therein and being rotatable therearound,
wherein the internally threaded collar segment comprises one or more internal threads configured to rotatably attach to the one or more external threads of the externally threaded cap.

9. The injection apparatus of claim 8, wherein the one or more external threads of the externally threaded cap comprises three external threads and the one or more internal threads of the internally threaded collar segment comprises three internal threads.

10. The injection apparatus of claim 9, wherein a rotation of between 220 degrees to 240 degrees secures the internally threaded collar segment to the externally threaded cap.

11. The injection apparatus of claim 10, wherein the cannula tip extends through the slit of the septum upon the rotation of the internally threaded collar segment, thereby fluidly connecting the locking cannula to the T-site body.

12. The injection apparatus of claim 8, wherein the externally threaded cap further comprises an inner flange extending distally from the proximal face, the inner flange defining a septum barrier wall abutting a septum wall of the septum.

13. The injection apparatus of claim 12, wherein the inner flange further defines an outer wall forming a pocket between the external wall and the outer wall, the outer wall comprising an annular protrusion extending outwardly into the pocket.

14. The injection apparatus of claim 13, wherein the septum housing comprises an internal recess configured to receive the annular protrusion.

15. A system for controlling flow of liquids to and from a patient, comprising:
a T-site connector comprising:
a T-site body extending in a longitudinal direction between a proximal end and a distal end,
wherein the proximal end comprises a septum housing and the distal end comprises a primary luer body having a tube bond stem disposed therebetween and extending axially in relation to the longitudinal direction;
a septum disposed within the septum housing, the septum comprising a slit therethrough;
an externally threaded cap disposed at the proximal end, the externally threaded cap comprising:
a proximal face having a bore therethrough; and
an external wall extending distally from the proximal face and having one or more external threads disposed therearound,
wherein the externally threaded cap is secured to the septum housing, thereby retaining the septum within the septum housing; and
a first internally threaded collar disposed at the primary luer body and being rotatable therearound;
a locking cannula comprising:
a cannula; and
a second internally threaded collar receiving a portion of the cannula therethrough, the second internally threaded collar comprising one or more internal threads configured to receive the one or more external threads of the externally threaded cap.

16. The system of claim 15, wherein a rotation of between 220 degrees to 240 degrees of the second internally threaded collar secures the second internally threaded collar to the externally threaded cap.

17. The system of claim 16, wherein the one or more internal threads of the second internally threaded collar comprises three internal threads and the one or more external threads of the externally threaded cap comprises three external threads.

18. The system of claim 17, wherein the septum comprises a flange extending radially outwards at a distal end of the septum, the flange being compressed between the externally threaded cap and the septum housing.

19. The system of claim 18, wherein the flange comprises a housing protrusion extending distally therefrom, further wherein the septum housing comprises an internal seat receiving the housing protrusion therein.

20. The system of claim 19, wherein the cannula comprises a cannula tip at a distalmost end and a cannula tip protrusion disposed proximally to the cannula tip, the cannula tip protrusion configured to abut a perimeter of the bore upon securing the second internally threaded collar to the externally threaded cap.
